(19) United States
(12) Patent Application Publication
(10) Pub. No.: US 2023/0066502 A1
(43) Pub. Date: Mar. 2, 2023

(54) PREDICTION METHOD FOR INDICATION OF AIMED DRUG OR EQUIVALENT SUBSTANCE OF DRUG, PREDICTION APPARATUS, AND PREDICTION PROGRAM

(71) Applicant: KARYDO THERAPEUTIX, INC., Tokyo (JP)

(72) Inventor: Narutoku Sato, Soraku-gun, Kyoto (JP)

(73) Assignee: KARYDO THERAPEUTIX, INC., Tokyo (JP)

(21) Appl. No.: 17/793,469

(22) PCT Filed: Jan. 15, 2021
(86) PCT No.: PCT/JP2021/001265

§ 371 Date: Jul. 18, 2022

(30) Foreign Application Priority Data
Jan. 17, 2020 (JP) 2020-006304

(51) Int. Cl. G16H 70/40; G06N 3/00

(52) U.S. Cl. CPC G16H 70/40; G06N 3/008

(57) ABSTRACT

An object of the present invention is to achieve prediction of an indication, drug repositioning and/or drug repurposing for a drug with no known adverse events and/or side effects based on adverse events and/or side effects.

The problem is solved by a method for predicting an indication for a drug of interest or its equivalent substance, including inputting estimated adverse event-related information and/or estimated side effect-related information estimated from a set of data indicating the behavior of a biomarker in one or more organs collected from non-human animals to which the drug of interest or its equivalent substance has been administered as a test substance into an artificial intelligence model for prediction as test data to predict an indication for the drug of interest or its equivalent substance.

### TECHNICAL FIELD

This specification discloses a method, a device, and a program for predicting an indication for a drug of interest or its equivalent substance.

### BACKGROUND ART

Discovery and development of a drug take a long time and a huge amount of money, and there are risks involved in the process. It is said that discovery and development of a new drug take an average of 12 years and cost about 2.6 billion dollars. Despite such tremendous effort, it is said that only 13.8% of drug candidates succeed in clinical trials. To avoid these problems, several strategies and approaches have been proposed and put into practice. One of them is repositioning and repurposing (DR) of existing drugs (Non-Patent Document 1).

DR is a method of exploring further therapeutic indication(s) (TI(s)) for clinically approved existing pharmaceutical products. In DR, the required development time is short and the cost is not as high as that for new drug development. Also, the pharmaceutical products have already been approved for use in treating at least one disease or symptom in humans. Thus, there is less concern about toxicity in humans. It is, therefore, possible in DR to skip the phase I clinical trials and proceed immediately to the phase II trials. In addition, because these drugs are already mass-produced for human use, the production process for clinical use has already been optimized. These characteristics of DR can lead to significant saving of time and cost in the development and approval processes (Non-Patent Document 1).

Currently, there are two main types of DR approaches. One of them is a method in which new indications and/or applications for each DR drug candidate are rationally designed and screened by thoroughly studying and understanding its biological, pharmacological, and/or structural properties. The other is a method depending on serendipity (incidental discovery). In other words, there may be the case where new indication and/or new applications are discovered incidentally during preclinical trials, clinical trials, and/or monitoring of new drugs in the real world. These general approaches are relatively ineffective and are the bottleneck of the current DR discovery process (Non-Patent Document 1).

As a method for assisting the exploration of candidate substances for new drugs in the development of a new drug, Patent Document 1 discloses a method including comparing test data of an organ-related index factor in each organ obtained from cells or tissues derived from one or more organs of individuals to which a test substance has been administered with preliminarily determined corresponding standard data of the organ-related index factor to obtain a pattern similarity for calculating the similarity of the pattern of the organ-related index factor, and predicting the efficacies or side effects of the test substance in the one or more organs and/or in organs other than the one or more organs using the pattern similarity of the organ-related index factor as an index.

Also, as a method for predicting efficacies or side effects of a candidate substance in the development of a new drug, Patent Document 2 and Non-Patent Document 2 disclose an artificial intelligence model for predicting one or more effects of a test substance on humans from the behavior of transcriptome in multiple different organs which are the same as those collected from non-human animals to which the test substance has been administered to prepare training data. The method includes inputting a data set indicating the behavior of transcriptome in multiple different organs collected from non-human animals to which multiple known drugs with known effects on humans have been individually administered for each of the non-human animals and data indicating known effects of each known drug on humans into the artificial intelligence model as training data to train the artificial intelligence model.

### RELATED ART DOCUMENT

### Patent Document

[Patent Document 1] WO2016/208776

[Patent Document 2] Japanese Paten No. 6559850

### Non-Patent Document

[Non-Patent Document 1] Pushpakom, S et al., (2019): Nature reviews Drug discovery 18, 41-58.

[Non-Patent Document 2] Kozawa, S et al., (2020): iScience (DOI: 10.1016/j.isci.2019.100791)

[Non-Patent Document 3] Li, J., and Lu, Z. (2012): Proceedings (IEEE Int Conf Bioinformatics Biomed) 2012, 1-4.

### SUMMARY OF THE INVENTION

### Problems to be Solved by the Invention

The method described in Non-Patent Document 3 is a method in which information about adverse events and/or side effects and information about indications are acquired from a known drug database to predict a new indication. In this case, the adverse events and/or side effects related to a drug of interest for which a new indication is desired to be explored must be known in advance. Thus, this method is not applicable to new drugs.

An object of the present invention is to achieve prediction of an indication, drug repositioning and/or drug repurposing for a drug with no known adverse events and/or side effects based on adverse events and/or side effects.

### Means for Solving the Problem

As a result of intensive studies, the present inventor found that prediction of an indication, drug repositioning and/or drug repurposing can be achieved for a drug with no known adverse events and/or side effects using an artificial intelligence model trained based on information about adverse events and/or side effects and information about indications for various known drugs registered in a public database or the like and an artificial intelligence model described in Patent Document 2 and Non-Patent Document 2.

The present invention has been made based on the finding, and includes the following aspects.

Embodiment 1. A method for predicting an indication for a drug of interest or its equivalent substance, including inputting estimated adverse event-related information estimated from a set of data indicating the behavior of a biomarker in one or more organs collected from non-human animals to which the drug of interest or its equivalent substance has been administered as a test substance into an artificial intelligence model for prediction as test data to predict an indication for the drug of interest or its equivalent substance.

Embodiment 2. The prediction method according to Embodiment 1, in which the artificial intelligence model for prediction is trained by means of a set of training data, and in which the set of training data is data in which (I) already reported adverse event-related information and/or already reported side effect-related information reported for individual known drugs is/are linked with (II) indication data reported for the known drugs.

Embodiment 3. The prediction method according to Embodiment 1 or 2, in which the artificial intelligence model for prediction corresponds to one indication.

Embodiment 4. The prediction method according to Embodiment 1 or 2, in which the artificial intelligence model for prediction corresponds to multiple indications.

Embodiment 5. The prediction method according to any one of Embodiments 1 to 4, in which the estimated adverse event-related information and/or estimated side effect-related information is/are generated using an artificial intelligence model for estimation that is different from the artificial intelligence model for prediction.

Embodiment 6. The prediction method according to any one of Embodiments 1 to 5, in which the set of training data is generated by linking labels indicating indications for the known drugs and information about adverse events reported for the known drugs with labels indicating the names of the known drugs.

Embodiment 7. The prediction method according to any one of Embodiments 1 to 6, in which the estimated adverse event-related information and/or estimated side effect-related information correspond(s) to (1) the presence or absence of multiple adverse events and/or side effects, or (2) the occurrence frequencies of multiple adverse events and/or side effects.

Embodiment 8. A device for predicting an indication for a drug of interest or its equivalent substance, including a processing part, in which the processing part is configured to input estimated adverse event-related information estimated from a set of data indicating the behavior of a biomarker in one or more organs collected from non-human animals to which the drug of interest or its equivalent substance has been administered as a test substance into an artificial intelligence model for prediction as test data to predict an indication for the drug of interest or its equivalent substance.

Embodiment 9. A computer program for predicting an indication for a drug of interest or its equivalent substance, executable by a computer to cause the computer to execute the step of inputting estimated adverse event-related information estimated from a set of data indicating the behavior of a biomarker in one or more organs collected from non-human animals to which the drug of interest or its equivalent substance has been administered as a test substance into an artificial intelligence model for prediction as test data to predict an indication for the drug of interest or its equivalent substance.

Embodiment 10. An estimation method for estimating an action mechanism of a test substance in a living organism, including hierarchizing the set of data indicating the behavior of a biomarker in one or more organs used in predicting an indication by clustering based on a prediction result about an indication predicted by a prediction method according to any one of Embodiments 1 to 7, and performing a pathway analysis on the hierarchized set of data indicating the behavior of a biomarker to acquire information about an action mechanism of the test substance.

Embodiment 11. An estimation device for estimating an action mechanism of a test substance in a living organism, including a processing part, in which the processing part is configured to hierarchize the set of data indicating the behavior of a biomarker in one or more organs used in predicting an indication by clustering based on a prediction result about an indication predicted by a prediction method according to any one of Embodiments 1 to 7, and to perform a pathway analysis on the hierarchized set of data indicating the behavior of a biomarker to acquire information about an action mechanism of the test substance.

Embodiment 12. An estimation program for estimating an action mechanism of a test substance in a living organism, executable by a computer to cause the computer to execute processing including the steps of: hierarchizing the set of data indicating the behavior of a biomarker in one or more organs used in predicting an indication by clustering based on a prediction result about an indication predicted by a prediction method according to any one of Embodiments 1 to 7, and performing a pathway analysis on the hierarchized set of data indicating the behavior of a biomarker to acquire information about an action mechanism of the test substance.

### Effect of the Invention

The present invention makes it possible to achieve prediction of an indication, drug repositioning and/or drug repurposing for a drug with no known adverse events and/or side effects based on adverse events and/or side effects.

### BRIEF DESCRIPTION OF DRAWINGS

FIG. 1 illustrates an overview of a method for predicting an indication disclosed in this specification.

FIG. 2 shows a method for estimating information about adverse events for generating test data.

FIG. 3 shows examples of training data. FIG. 3(A) shows an example of a set of training data for nerve injury. FIG. 3(B) shows a set of training data for type 2 diabetes mellitus.

FIG. 4 shows a hardware configuration of a training device 10 for prediction.

FIG. 5 shows a flowchart of training processing for prediction.

FIG. 6 shows an example of data indicating the behavior of a biomarker.

FIG. 7 shows an example of generated second training data.

FIG. 8 illustrates a hardware configuration of a device 50 for generating test data for prediction.

FIG. 9 shows a flowchart of processing by a training program for estimation.

FIG. 10 shows a flowchart of processing by an estimation program.

FIG. 11 illustrates a hardware configuration of a prediction device 20.

FIG. 12 shows a flowchart of prediction processing.

FIG. 13 illustrates a hardware configuration of a device 80 for estimating an action mechanism.

FIG. 14 shows a flowchart of processing by an analysis program.

FIG. 15 shows distributions of accuracy, recall and precision scores for all drugs.

FIG. 16 shows respective scores of the top 50 drugs having accuracy, precision and recall scores that are all 1.0 among drugs for which indication prediction was performed.

FIG. 17 shows distributions of accuracy, recall and precision scores for all indications.

FIG. 18 shows respective scores of the top 50 indications having accuracy, precision and recall scores that are all 1.0 among predicted indications.

FIG. 19 shows results of blind evaluation.

FIG. 20 shows comparison between V-AE and R-AE.

FIG. 21 shows indication prediction results for 15 test drugs obtained using V-AE. FIG. 21(A) shows results of mixed matrix. FIG. 21(B) shows comparison of accuracy, precision and recall scores between indication prediction results for 15 test drugs obtained using V-AE and those obtained using LP.

FIG. 22 shows comparison between indication prediction results by V-AE and indication prediction results by One-Class SVM using R-AE. The upper part shows comparison of TP, and the lower part shows comparison of FP.

FIG. 23 shows comparison between indication prediction results by V-AE and indication prediction results by LP using R-AE. The upper part shows comparison of TP, and the lower part shows comparison of FP.

FIG. 24(A) is a tree diagram showing the relationship between V-AE of each test drug and each indication. FIG. 24(B) is a tree diagram showing the relationship between a transcriptome profile of each test drug and each indication.

FIG. 25 shows comparison between action mechanisms of drugs for osteoporosis and schizophrenia. FIG. 25(A) shows distribution of V-AE, and FIG. 25(B) shows distribution of transcriptome patterns.

FIG. 26 shows results of comparison between pathways associated with the effects of drugs on osteoporosis and schizophrenia in each organ that were predicted using REACTOME Pathways.

FIG. 27 shows results of comparison between pathways associated with the effects of drugs on osteoporosis and schizophrenia in each organ that were predicted using KEGG pathway.

### DETAILED DESCRIPTION OF THE INVENTION

### 1. Overviews of Training Method and Prediction Method, and Description of Terms

First, a method for training an artificial intelligence and a prediction method as certain embodiments of this disclosure are outlined. The prediction method predicts an indication for a drug of interest or its equivalent substance (in this specification, a drug and its equivalent substance may be collectively referred to simply as “drug or the like”). Preferably, the prediction method uses as test data information related to adverse events (AEs) and/or information related to side effects (SEs) estimated from the behavior of a biomarker (which are hereinafter referred to as “estimated adverse event-related information” and “estimated side effect-related information,” respectively) obtained by administering a drug of interest or its equivalent substance to non-human animals as a test substance, collecting one or more organs from the drug-administered non-human animals, and acquiring a set of data indicating the behavior of a biomarker from the one or more organs collected. The prediction method predicts an indication (therapeutic indication: TI) of the drug of interest or its equivalent substance based on the test data. The prediction is achieved using artificial intelligence models. Here, for convenience sake, an example using adverse events is shown.

### (1) Training Phase

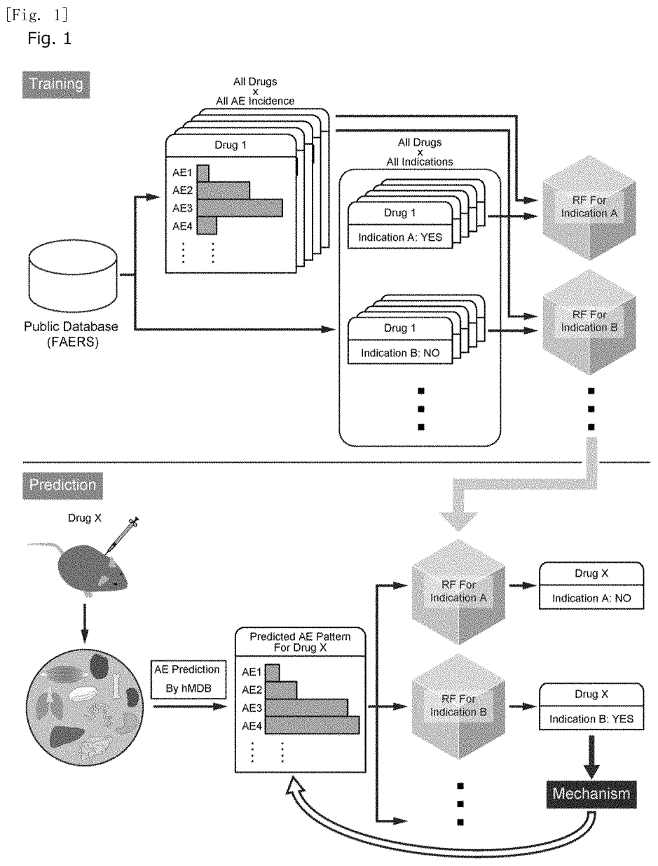

The upper part of FIG. 1 shows an overview of a training phase. Training data includes information about adverse events in humans reported for known drugs (which may be hereinafter referred to also as “already reported adverse event-related information”) and indication data reported for the known drugs based on information available from a public drug database. FAERS, which is described later, is shown as an example in FIG. 1, and adverse events reported and unreported in humans are registered for each drug in this drug database. In other words, information about whether or not each of multiple adverse events has appeared is registered for each drug. In this specification, information about whether or not a certain adverse event has appeared (the presence or absence of a certain adverse event) for one drug is referred to as adverse event data. Adverse event data is linked with a label indicating a drug name that indicates to which drug the adverse event data belongs. In the drug database, multiple items of adverse event data are registered per drug, and these constitute a set of adverse event data. Thus, the information about adverse events may include (i) a set of adverse event data registered for one drug, or (ii) a set of occurrence frequency data for each adverse event calculated based on a set of adverse event data for one drug. The occurrence frequency data is linked with a label indicating a drug name that indicates to which drug the occurrence frequency data belongs.

Similarly, for indications as well, applicable diseases or symptoms, and diseases or symptoms in humans for which applicability has not been reported are registered for each drug. In other words, for multiple diseases or symptoms, information indicating whether or not each disease or symptom is an indication is registered for each drug. In this specification, information indicating whether or not one drug may be applicable to a certain disease or symptom is referred to as “indication data.” Indication data is linked with a label indicating a drug name that indicates to which drug the indication data belongs. In a drug database, multiple items of indication data are registered per drug, and these constitute a set of indication data. The information indicating whether or not a disease or symptom is an indication that is included in the training data is merely information registered in a drug database and may include information that has not been experimentally confirmed if the drug is actually applicable.

Here, the term “linked” is merely intended to mean that a label is attached so that the correspondence relationship between each item of data and a drug to which the data belongs can be understood. No label indicating a drug name is attached to the information about adverse events and the indication data to be input into an artificial intelligence.

In the upper part of FIG. 1, pieces of information about adverse events (AE1, AE2, AE3, AE4, . . . in FIG. 1) reported for individual known drugs (Drug 1, . . . in FIG. 1) can be linked with each item of indication data (Indication A: YES, Indication B: NO) for each drug based on, for example, labels indicating the drug names.

By way of example, FIG. 1 shows an example in which artificial intelligence models that do not have a neural network structure such as random forests (RFs) are used.

In this example, one artificial intelligence model is used for one indication, and an artificial intelligence model is trained for each indication.

Thus, in order to predict the applicability to a predetermined indication (for example, Indication A), pieces of information about adverse events reported for individual known drugs (AE1, AE2, AE3, AE4, . . . in FIG. 1), and indication data corresponding to each drug (for example, Indication A: YES) are input in combination into one artificial intelligence model to train the artificial intelligence model. Similarly, in order to predict the applicability to another indication (for example, Indication B), pieces of information about adverse events reported for individual known drugs (AE1, AE2, AE3, AE4, . . . in FIG. 1), and indication data corresponding to each drug (for example, Indication B: No) are input in combination into one artificial intelligence model to train the artificial intelligence model. The artificial intelligence models trained in this training phase are artificial intelligence models for predicting an indication from test data for prediction as described later, and are referred to as artificial intelligence models for prediction.

The drugs may or may not include drugs for which test data that is used in the prediction phase is acquired.

### (2) Prediction Phase

Next, the trained artificial intelligence models are used to predict an indication for a drug of interest or its equivalent substance. Preferably, an indication in humans is predicted. More preferably, a new indication is predicted. A new indication is an indication that has not been known for a certain drug.

Test data for prediction is generated according to the method described in Patent Document 2 and Non-Patent Document 2. Specifically, test data for prediction is generated using an artificial intelligence model for estimation that is different from the artificial intelligence model for prediction.

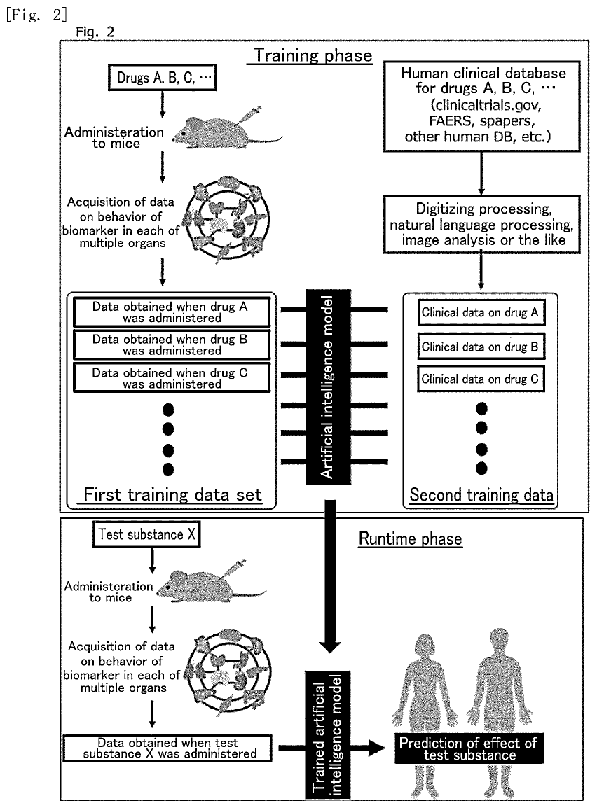

FIG. 2 shows an overview of a method for training an artificial intelligence model for estimation to generate test data for prediction, and a method for generating test data for prediction using an artificial intelligence model for estimation.

As shown in FIG. 2, in a training phase for an artificial intelligence model for estimation, known drugs A, B and C, for example, are administered individually to non-human animals such as mice, and an organ or a tissue as a part of an organ is collected from the respective non-human animals. Next, the behavior of a biomarker in the collected organs or tissues is analyzed to generate a first training data set reflecting the behavior of a biomarker. Also, second training data, which is information about adverse events, is generated from a human clinical database (drug database) storing information about adverse events reported for known drugs.

The artificial intelligence model for estimation is generated by training an artificial intelligence model for estimation using the first training data set and the second training data. An estimation phase predicts adverse events related to a test substance X in humans by means of a trained artificial intelligence model for estimation using data indicating the behavior of a biomarker in one or more organs of non-human animals to which the test substance X has been administered as test data for estimation. Specifically, one or more organs or part of an organ is/are individually collected from non-human animals to which the test substance X has been administered to acquire a set of data indicating the behavior of a biomarker in each organ. Subsequently, the data set is input into the trained artificial intelligence model for estimation as test data for estimation to predict the presence or absence of adverse events related to the test substance X in humans or the occurrence frequency thereof. The (A) set of data on adverse events predicted for the test substance X or (B) the set of data on occurrence frequency of each adverse event predicted for the test substance X output from the artificial intelligence model for estimation serves as estimated adverse event-related information estimated for the test substance X. The set of data on adverse events and data on occurrence frequency are linked with labels indicating drug names that indicate the drug to which the occurrence frequency data belongs. In this way, respective data can be acquired according to a method described in Patent Document 2 and Non-Patent Document 2, and information about adverse events can be estimated using these data for a drug for which no adverse event is registered in a known drug database.

Referring again to FIG. 1, a prediction phase in which an indication for a drug or the like of interest is predicted using artificial intelligence models for prediction is described. In the prediction phase, estimated adverse event-related information estimated by an artificial intelligence model for estimation is used as test data. The test data is input into artificial intelligence models trained as described in Section (1) above to predict an indication.

The lower part of FIG. 1 shows an example of a prediction phase. Here, based on a set of data indicating the behavior of a biomarker in each organ acquired from non-human animals to which a drug (drug X) for which an indication is desired to be predicted has been administered, pieces of information AE1, AE2, AE3, AE4, . . . about estimated adverse events are generated using an artificial intelligence model for estimation according to the above-mentioned method. The “hMDB” described in the lower part of FIG. 1 is intended to mean humanized Mouse DataBase individualized, hMDB-i reported in Non-Patent Document 2. The pieces of information AE1, AE2, AE3, AE4, . . . about estimated adverse events are respectively input as test data for prediction into artificial intelligence models trained for each indication (RF for Indication A, and RF for Indication B in FIG. 1). When the drug X is not effective against Indication A, a label “NO” indicating that there is no applicability is output from the RF for Indication A, which predicts applicability to Indication A. On the other hand, when the drug X is effective against Indication B, a label “YES” is output from the RF for Indication B. At this time, Indication B can be predicted to be an indication for the drug X. When Indication B is an indication that has not been known for the drug X, Indication B is a new indication for the drug X.

In this way, by using hMDB, it is possible to predict an indication in humans for a drug or the like for which adverse events are not registered in a known drug database based on information about adverse events.

Further, this embodiment includes predicting an action mechanism of a drug or the like of interest from the predicted indication.

### (3) Description of Terms

In this disclosure, the term “drug” includes pharmaceutical products, quasi-pharmaceutical products, cosmeceutical products, foods, foods for specified health use, foods with functional claims and candidates therefor. Also, the term “drug” also includes substances whose testing was discontinued or suspended during a preclinical or clinical trial for pharmaceutical approval. Also, the term “drug” includes new drugs and known drugs. More specifically, the term “drug” may include, for example, compounds; nucleic acids; carbohydrates; lipids; glycoproteins; glycolipids; lipoproteins; amino acids; peptides; proteins; polyphenols; chemokines; at least one metabolic substance selected from the group consisting of ultimate metabolites, intermediary metabolites and synthetic raw material substances of the above-mentioned substances; metal ions; or microorganisms. Here, the term “drug” or its equivalent substance may include single drugs and companion drugs in which multiple drugs are combined.

The “drug of interest” is a drug for which an indication is desired to be predicted.

The “known drug” is not limited as long as it is an existing drug. Preferably, it is a drug with known effects on humans. Also, the term “equivalent substance of a drug” may include drugs that have a similar structure and a similar effect to an existing drug. The term “similar effect” here is intended to mean having the same kind of effect as a known drug although the intensity of the effect is different.

The “adverse event” is not limited as long as it is an effect that is determined to be harmful to humans. Preferred examples include adverse events listed in public drug databases such as FAERS

(https://www.fda.gov/Drugs/GuidanceComplianceRegulatoryInformation/Surveillance/Adverse DrugEffects/ucm082193.htm) or clinicaltrials.gov (https://clinicaltrials.gov/).

The term “side effect” is intended to mean an effect on humans other than the indication for each drug, not limited to adverse events. Examples of the side effect include those listed in a public drug database such as SIDER4.1 (http://sideeffects.embl.de).

The occurrence frequency of an adverse event or side effect can be obtained by the following method. A word or phrase indicating the name of an adverse event is extracted by, for example, text extraction from a database as described above such as clinicaltrials.gov, FAERS, or all drug labels of DAILYMED. One extracted word or phrase can be counted as one reported adverse event. When an adverse event is taken as an example, for one known drug, the occurrence frequency can be obtained according to the equation: Occurrence frequency=(the number of cases reported for one adverse event)/(the total number of cases of adverse events reported for the known drug). When explanations related to effects are registered in text form in a database, syntactic analysis, word segmentation, semantic analysis or the like may be performed on the registered texts by natural language processing before the extraction of the texts corresponding to the effects.

The “indication” is not limited as long as it is a disorder or symptom in humans that should be mitigated, treated, arrested or prevented. Examples of the disorder or symptom include disorders or symptoms listed in a public drug database such as the above-mentioned FAERS, all drug labels of DAILYMED (https://dailymed.nlm.nih.gov/dailymed/spl-resources-all-drug-labels.cfm), Medical Subject Headings (https://www.nlm.nih.gov/mesh/meshhome.html), Drugs@FDA (https://www.accessdata.fda.gov/scripts/cder/daf/), or International Classification of Diseases (https://www.who.int/health-topics/international-classification-of-diseases). More specifically, examples of the indication include ischemic disorders such as thrombosis, embolism and stenosis (in particular, heart, brain, lungs, large intestine, etc.); circulatory disorders such as aneurysm, phlebeurysm, congestion and hemorrhage (aortae, veins, lungs, liver, spleen , retinae, etc.); allergic diseases such as allergic bronchitis and glomerulonephritis; dementia such as Alzheimer's dementia; degenerative disorders such as Parkinson's disease, amyotrophic lateral sclerosis and myasthenia gravis (nerves, skeletal muscles, etc.); tumors (benign epithelial tumor, benign non-epithelial tumor, malignant epithelial tumor, malignant non-epithelial tumor); metabolic diseases (abnormal carbohydrate metabolism, abnormal lipid metabolism, electrolyte imbalance); infectious diseases (bacteria, viruses, rickettsia, chlamydia, fungi, protozoa, parasite, etc.); and symptoms or illnesses associated with autoimmune diseases or the like such as renal diseases, systemic erythematodes and multiple sclerosis.

In this disclosure, the term “artificial intelligence model” means a unit of algorithms that can output a result of interest from a set of input data. Examples of the artificial intelligence model may include random forest (RF), support vector machine (SVM), relevance vector machine (RVM), naive Bayes, logistic regression, feedforward neural network, deep learning, K-nearest neighbor algorithm, AdaBoost, bagging, C4.5, Kernel approximation, stochastic gradient descent (SGD) classifier, Lasso, ridge regression, elastic net, SGD regression, kernel regression, LOWESS regression, matrix fractorization, nonnegative matrix fractorization, kernel matrix fractorization, interpolation, kernel smoother, and collaborative filtering.

In this disclosure, training an artificial intelligence model for prediction and an artificial intelligence model for estimation may include validation, generalization or the like. Examples of the validation and generalization include holdout method, cross-validation method, AIC (An Information Theoretical Criterion/Akaike Information Criterion), MDL (Minimum Description Length), and WAIC (Widely Applicable Information Criterion).

In this disclosure, the non-human animals are not limited. Examples include mammals such as mice, rats, dogs, cats, rabbits, cows, horses, goats, sheep and pigs, and birds such as chickens. Preferably, the non-human animals are mammals such as mice, rats, dogs, cats, cows, horses and pigs, more preferably mice, rats or the like, and still more preferably mice. The non-human animals also include fetuses, chicks and so on of the animals.

The “organ” is not limited as long as it is an organ present in the body of a mammal or bird as described above. For example, in the case of a mammal, the organ is at least one selected from circulatory system organs (heart, artery, vein, lymph duct, etc.), respiratory system organs (nasal cavity, paranasal sinus, larynx, trachea, bronchi, lung, etc.), gastrointestinal system organs (lip, cheek, palate, tooth, gum, tongue, salivary gland, pharynx, esophagus, stomach, duodenum, jejunum, ileum, cecum, appendix, ascending colon, transverse colon, sigmoid colon, rectum, anus, liver, gallbladder, bile duct, biliary tract, pancreas, pancreatic duct, etc.), urinary system organs (urethra, bladder, ureter, kidney), nervous system organs (cerebrum, cerebellum, mesencephalon, brain stem, spinal cord, peripheral nerve, autonomic nerve, etc.), female reproductive system organs (ovary, oviduct, uterus, vagina, etc.), breast, male reproductive system organs (penis, prostate, testicle, epididymis, vas deferens), endocrine system organs (hypothalamus, pituitary gland, pineal body, thyroid gland, parathyroid gland, adrenal gland, etc.), integumentary system organs (skin, hair, nail, etc.), hematopoietic system organs (blood, bone marrow, spleen, etc.), immune system organs (lymph node, tonsil, thymus, etc.), bone and soft tissue organs (bone, cartilage, skeletal muscle, connective tissue, ligament, tendon, diaphragm, peritoneum, pleura, adipose tissue (brown adipose, white adipose), etc.), and sensory system organs (eyeball, palpebra, lacrimal gland, external ear, middle ear, inner ear, cochlea, etc.). Preferably, the “organ” is at least one selected from bone marrow, pancreas, skull bone, liver, skin, brain, brain pituitary gland, adrenal gland, thyroid gland, spleen, thymus, heart, lung, aorta, skeletal muscle, testicle, epididymal fat, eyeball, ileum, stomach, jejunum, large intestine, kidney, and parotid gland. Preferably, all of bone marrow, pancreas, skull bone, liver, skin, brain, brain pituitary gland, adrenal gland, thyroid gland, spleen, thymus, heart, lung, aorta, skeletal muscle, testicle, epididymal fat, eyeball, ileum, stomach, jejunum, large intestine, kidney, and parotid gland are used in the prediction according to this disclosure. The term “multiple organs” is not limited as long as the number of organs is two or more. For example, the multiple organs can be selected from 2, 3, 4, 5, 6, 7, 8, 9, 10, 11, 12, 13, 14, 15, 16, 17, 18, 19, 20, 21, 22, 23, or 24 types of organs.

The term “biomarker” means a biological substance that can be varied in the cells or tissues of each organ and/or in a body fluid depending on the administration of the substance. An example of a biological substance that may serve as a “biomarker,” is at least one selected from nucleic acids; carbohydrates; lipids; glycoproteins; glycolipids; lipoproteins; amino acids, peptides; proteins; polyphenols; chemokines; at least one metabolic substance selected from the group consisting of ultimate metabolites, intermediary metabolites and synthetic raw material substances of the above-mentioned substances; metal ions and so on. More preferred is a nucleic acid. The biomarker is preferably a group of biological substances that are varied in the cells or tissues of each organ and/or in a body fluid depending on the administration of the substance. An example of a group of biological substances is a group of at least one kind selected from nucleic acids; carbohydrates; lipids; glycoproteins; glycolipids; lipoproteins; amino acids, peptides; proteins; polyphenols; chemokines; at least one metabolic substance selected from the group consisting of ultimate metabolites, intermediary metabolites and synthetic raw material substances of the above-mentioned substances; metal ions and so on.

The term “nucleic acids” preferably means a group of RNAs contained in transcriptome, such as mRNAs, non-coding RNAs and microRNAs, more preferably a group of mRNAs. The RNAs are preferably mRNAs, non-coding RNAs and/or microRNAs that may be expressed in the cells or tissues of the above organs or cells in a body fluid, more preferably mRNAs, non-coding RNAs and/or microRNAs that may be detected by RNA-Seq or the like

(https://www.ncbi.nlm.nih.gov/gene?LinkName=genome_gene&from_uid=52, http://jp.support.illumina.com/sequencing/sequencing_software/igenome.html). Preferably, all RNAs that can be analyzed as RNA-Seq are used for the prediction according to this disclosure.

The term “a set of data indicating the behavior of a biomarker” is intended to means a set of data indicating that the biomarker has or has not been varied in response to the administration of a drug or the like. Preferably, the behavior of a biomarker indicates that the biomarker has been varied in response to the administration of a drug or the like. The data can be acquired by, for example, the following method. For tissues, cells, body fluids or the like derived from certain organs collected from non-human animals to which a drug or the like has been administered, the abundance or concentration of each biomarker is measured to acquire a measurement value for each organ of the individuals to which the drug or the like has been administered. Also, from non-human animals to which the drug or the like has not been administered, the abundance or concentration of each biomarker is measured for tissues, cells, body fluids or the like derived from organs corresponding to the organs from which measurement values of the individuals to which the drug or the like has been administered were acquired in the same manner to acquire measurement values in non-administered individuals. The measurement values of each biomarker derived from each organ of the individuals to which the drug or the like has been administered are compared with the measurement values in non-administered individuals of the biomarker for each organ corresponding to the biomarkers in the individuals to which the drug or the like has been administered to acquire values indicating the differences therebetween as data. Here, the term “corresponding to” means that the organs and biomarkers are the same or of the same type. Preferably, the differences can be represented as ratios (such as quotients) of the measurement values of respective biomarkers derived from the individuals to which the drug or the like has been administered to the measurement values of biomarkers corresponding to the above biomarkers in the non-administered individuals. For example, the data includes quotients obtained by dividing the measurement values of biomarker A in organs A derived from individuals to which the drug or the like has been administered by the measurement values of biomarker A in organs A derived from non-administered individuals.

When the biomarker is transcriptome, all RNAs that can be analyzed by RNA-Seq may be used. Alternatively, the RNAs may be analyzed for their expression, and divided into subsets (modules) of data indicating the behavior of each RNA with which the organ name and the gene name are linked using, for example, WGCNA

(https://labs.genetics.ucla.edu/horvath/CoexpressionNetwork/Rpackages/WGCNA/). For each module divided by means of WGCNA, a Pearson's correlation coefficient with 1-of-K representation may be calculated for each drug or the like to select a module with the highest absolute value of the correlation coefficient for each drug or the like, and the RNA in each organ included in the selected module may be used as a biomarker.

Further, when the biomarker in response to the administration of a drug or the like is transcriptome, the variation in transcriptome in each organ of the animals to which the drug or the like has been administered compared with that of the animals to which the drug or the like has not been administered can be obtained using DESeq2 analysis. For example, the expression levels of RNAs in each organ collected from animals to which the drug or the like has been administered and the expression levels of genes in each corresponding organ collected from animals to which the drug or the like has not been administered are quantified by htseq-count to obtain count data of respective organs. Then, respective organs and the expression levels of respective genes in respective organs are compared. As a result of the comparison, a loge (fold) value of the variation in gene expression in the animals to which the drug or the like has been administered and a p-value, which serves as an index of the probability of each variation, are output for each gene in each organ. Based on the loge (fold) value, it is possible to determine whether or not the behavior of a biomarker such as transcriptome is present.

The term “organ-derived” is intended to mean, for example, being collected from an organ, or being cultured from cells, tissues or a body fluid of a collected organ.

The term “body fluid” includes, for example, serum, plasma, urine, spinal fluid, ascites, pleural effusion, saliva, gastric juice, pancreatic juice, bile, milk, lymph and intercellular fluid.

The measurement values of a biomarker can be acquired by a known method. When the biomarker is a nucleic acid, the measurement values can be acquired by sequencing such as RNA-Seq, quantitative PCR, or the like. When the biomarker is a carbohydrate, lipid, glycolipid, amino acid, polyphenol; chemokine; at least one metabolic substance selected from the group consisting of ultimate metabolites, intermediary metabolites and synthetic raw material substances of the above-mentioned substances or the like, the measurement values can be acquired by, for example, mass spectrometry. When the biomarker is a glycoprotein, lipoprotein, peptide, protein or the like, the measurement values can be acquired by, for example, an ELISA (Enzyme-Linked Immuno Sorbent Assay) method. The method for collecting tissues, cells or body fluids derived from organs for use in the measurement and the preprocessing method for the measurement of a biomarker are also known.

The “test substance” is a substance to be evaluated for its effects. The test substance may be a drug or an equivalent of a drug. The test substance may be an existing substance or a new substance. In the prediction method, even when the relationship between an effect of the test substance and an effect of a known drug or an equivalent of a known drug has not been found, it is possible to predict an effect of the test substance on humans. On the other hand, when the test substance is one selected from known drugs or equivalents of known drugs, at least one unknown effect of the known drug or an equivalent of the known drug can be found. The at least one unknown effect may be one effect or multiple effects. The at least one unknown effect is preferably a new indication. By predicting a new indication for a test substance in humans, drug repositioning can be also achieved. Administration of a test substance to non-human animals is known. Also, the data indicating the behavior of a biomarker in one or more organs collected from non-human animals to which a test substance has been administered can be acquired in the same manner as the data indicating the behavior of a biomarker in one or more organs collected from non-human animals to which a drug or the like has been administered.

### 2. Construction of Artificial Intelligence Model For Prediction

Construction of an artificial intelligence model for prediction is described using adverse events as an example.

### 2-1. Generation of Training Data

A method for generating training data is described. The training data includes already reported adverse event-related information and indication data reported for the known drugs, which are generated based on information available from a public drug database 60.

For the definition of the terms “adverse event data,” “information about adverse event,” and “indication data,” the description in Section 1.(1) above is incorporated here.

Some drug databases, such as FAERS, basically include both adverse event data and indication data for each drug. In such a case, adverse event data reported for known drugs and indication data reported for the known drugs can be acquired from one drug database.

On the other hand, because only information about adverse events is described in, for example, clinicaltrials.gov or the like, the indications for each drug can be obtained from another drug database, such as FAERS, all drug labels of DAILYMED, Medical Subject Headings, Drugs@FDA, International Classification of Diseases or the like.

As described in Section 1.(1) above, the adverse event data and indication data registered in a drug database are linked with labels indicating drug names so that one can understand to which drug each item of data belongs. The labels may be the drug names themselves or may be the registration numbers or the like of the drugs.

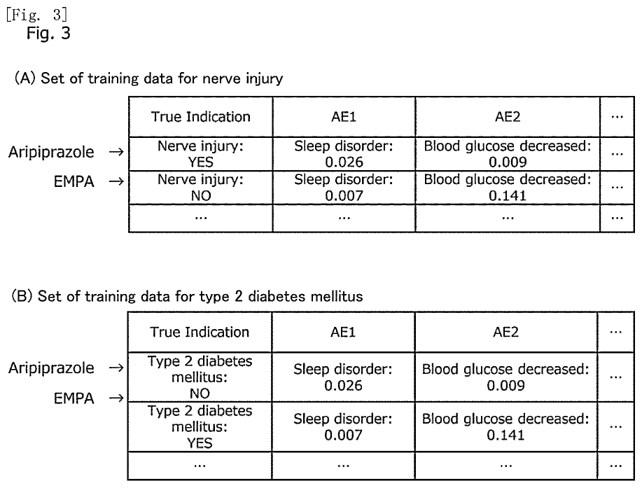

FIG. 3 shows examples of training data. FIG. 3(A) shows an example of a set of training data for nerve injury, and FIG. 3(B) shows a set of training data for type 2 diabetes mellitus. The names, such as Nerve injury and Type 2 diabetes mellitus, serve as labels indicating indication names. In FIG. 3, aripiprazole and empagliflozin (EMPA) are shown as examples of known drugs. Aripiprazole and EMPA serve as labels indicating drug names. In FIG. 3, “True Indication” is intended to mean an indication against which the drug has been proved to be effective that is registered in a drug database. For example, “True Indication” is nerve injury in FIG. 3(A), and “True Indication” is type 2 diabetes mellitus in FIG. 3(B). Because aripiprazole is a drug that is applicable to nerve injury, “Nerve injury: YES” has been entered in the column of “True Indication” in FIG. 3(A). Because EMPA is a drug that is not applicable to nerve injury, “Nerve injury: NO” has been entered in the column of “True Indication.” Because aripiprazole is a drug that is not applicable to type 2 diabetes mellitus, “Type 2 diabetes mellitus: NO” has been entered in the column of “True Indication” in FIG. 3(B). Because EMPA is a drug that is applicable to type 2 diabetes mellitus, “Type 2 diabetes mellitus: YES” has been entered in the column of “True Indication.”

“Nerve injury: YES,” “Nerve injury: NO,” “Type 2 diabetes mellitus: NO,” and “Type 2 diabetes mellitus: YES” serve as items of indication data.

The labels indicating whether or not a drug is effective against an indication that have been registered in a drug database may be “Y” and “N,” “1” and “0,” “1” and “−1” or the like besides “YES” and “NO.”

As described in Section 1.(1) above, multiple items of indication data are registered per drug in a drug database, and these constitute a set of indication data.

In FIG. 3, Sleep disorder and Blood glucose decreased are shown as examples of adverse events. In FIG. 3(A), “Sleep disorder: 0.026” and “Blood glucose decreased: 0.009” are contained in the row of aripiprazole. The values “0.026” and “0.009” represent the occurrence frequencies of the respective adverse events. Thus, “Sleep disorder: 0.026” and “Blood glucose decreased: 0.009” serve as occurrence frequency data for the respective adverse events. Thus, “Sleep disorder: 0.026” and “Blood glucose decreased: 0.009” constitute already reported adverse event-related information about aripiprazole. Thus, in the row of aripiprazole in FIG. 3(A), “Nerve injury: YES” as indication data is linked with “Sleep disorder: 0.026” and “Blood glucose decreased: 0.009” as already reported adverse event-related information. In other words, the combination of “Nerve injury: YES” with “Sleep disorder:0.026” and “Blood glucose decreased: 0.009” linked therewith (which may be represented as [“Nerve injury: YES”_“Sleep disorder: 0.026″+”Blood glucose decreased: 0.009″]) constitutes one item of training data.

Also, in FIG. 3(A), “Sleep disorder: 0.007” and “Blood glucose decreased: 0.141” are contained in the row of EMPA. “Sleep disorder: 0.007” and “Blood glucose decreased: 0.141” constitute already reported adverse event-related information about EMPA. Thus, a combination in which indication data “Nerve injury: NO” is linked with these pieces of already reported adverse event-related information (which may be represented as [“Nerve injury: NO”_“Sleep disorder: 0.007″+”Blood glucose decreased: 0.141″]) constitutes one item of training data.

In FIG. 3(B), “Sleep disorder: 0.026” and “Blood glucose decreased: 0.009” are contained as already reported adverse event-related information in the row of aripiprazole. In FIG. 3(B), indication data for aripiprazole is “Type 2 diabetes mellitus: NO.” The combination of “Type 2 diabetes mellitus: NO” with the already reported adverse event-related information (which may be represented as [“Type 2 diabetes mellitus: NO”_“Sleep disorder: 0.026”+“Blood glucose decreased: 0.009”]) constitutes one item of training data.

In FIG. 3(B), “Sleep disorder: 0.007” and “Blood glucose decreased: 0.141” are contained as already reported adverse event-related information in the row of EMPA. In FIG. 3(B), indication data for aripiprazole is “Type 2 diabetes mellitus: YES.” The combination of “Type 2 diabetes mellitus: NO” with the already reported adverse event-related information (which may be represented as [“Nerve injury: YES”_“Sleep disorder: 0.007”+“Blood glucose decreased: 0.141”] constitutes one item of training data.

When the artificial intelligence models for prediction are artificial intelligence models that do not have a neural network structure such as support vector machines (SVMs), one artificial intelligence model is used for one indication, and one artificial intelligence model is trained for each indication. Thus, a set of training data includes [“Nerve injury: YES”_“Sleep disorder: 0.026”+“Blood glucose decreased: 0.009”] and [“Nerve injury: NO”_“Sleep disorder: 0.007”+“Blood glucose decreased: 0.141”].

When the artificial intelligence models for prediction are artificial intelligence models having a neural network structure, one artificial intelligence model is trained for multiple indications. In other words, one trained artificial intelligence model corresponds to prediction of multiple indications. Thus, a set of training data includes [“Nerve injury: YES”+“Nerve injury: NO”_“Sleep disorder: 0.026”+“Blood glucose decreased: 0.009”] and [“Type 2 diabetes mellitus: NO”+“Type 2 diabetes mellitus: YES”_“Sleep disorder: 0.026”+“Blood glucose decreased: 0.009”]. The set of training data for artificial intelligence models having a neural network structure is not limited as long as already reported adverse event-related information about multiple drugs is associated with a set of indication data for the multiple drugs.

For convenience sake, two drugs and two adverse events are shown as examples in FIG. 3, and two items of indication data are respectively shown in FIG. 3(A) and FIG. 3(B) as examples. To increase predictable indications, it is preferred to use as many drugs as possible and adverse events data and indication data corresponding thereto.

The drug is not limited as long as it is a drug with which adverse event data and indication data are linked in a drug database as described above. The number of drugs is preferably 1,000 or more, 2,000 or more, 3,000 or more, or 4,000 or more. The upper limit is the number of drugs registered in the drug database.

The number of items of indication data registered per drug is preferably 1,000 or more, 5,000 or more, or 10,000 or more. The upper limit is the number of items of indication data registered in the drug database.

The number of items of adverse event data registered per drug is preferably 1,000 or more, 5,000 or more, or 10,000 or more. The upper limit is the number of items of adverse event data registered in the drug database.

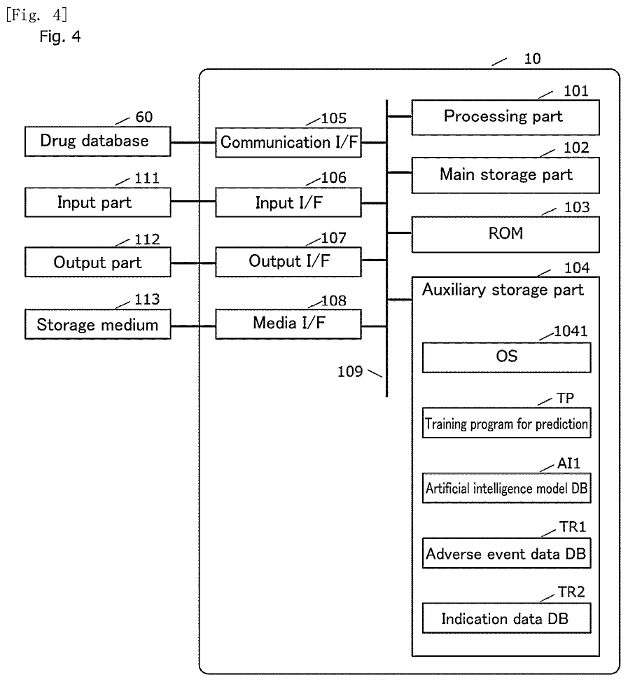

For the acquisition of adverse event data or a set of adverse event data from the drug database 60 shown in FIG. 4, a processing part 101 of a training device 10 starts the acquisition via a communication I/F 105 when the processing part 101 accepts a request to acquire data from an operator. The adverse event data or the set of adverse event data acquired are recorded in an adverse event database (DB) TR1 stored in an auxiliary storage part 104 by the processing part 101. Also, for the acquisition of indication data and a set of indication data from the drug database 60 shown in FIG. 4 as well, the processing part 101 of the training device 10 starts the acquisition via the communication I/F 105 when the processing part 101 accepts a request to acquire data from the operator. The indication data and the set of indication data acquired are recorded in a database (DB) TR2 for indication data of the auxiliary storage part 104 shown in FIG. 4 by the processing part 101.

### 2-2. Device for Training Artificial Intelligence Model For Prediction

The training of an artificial intelligence model for prediction as described above can be achieved using, for example, the training device 10 (which is hereinafter referred to also as “device 10”).

In the description of the device 10 and the processing in the device 10, for the terms that are common to those described in Sections 1. and 2-1. above, the above description is incorporated here.

FIG. 4 illustrates a hardware configuration of the device 10. The device 10 includes at least the processing part 101 and a storage part. The storage part is constituted of a main storage part 102 and/or an auxiliary storage part 104. The device 10 may be connected to an input part 111, an output part 112, and a storage medium 113. Also, the device 10 is communicably connected to a drug database 60 such as FAERS, all drug labels of DAILYMED, Medical Subject Headings, Drugs@FDA, International Classification of Diseases, or clinicaltrials.gov.

In the device 10, the processing part 101, the main storage part 102, a ROM (read only memory) 103, the auxiliary storage part 104, the communication interface (I/F) 105, an input interface (I/F) 106, an output interface (I/F) 107, and a media interface (I/F) 108 are connected for mutual data communication by a bus 109.

The processing part 101 is constituted of a CPU, MPU, GPU or the like. The processing part 101 executes a computer program stored in the auxiliary storage part 104 or the ROM 103 and processes the acquired data, whereby the device 10 functions. The processing part 101 trains an artificial intelligence model for prediction using training data as described in Section 1. above.

The ROM 103 is constituted of a mask ROM, a PROM, an EPROM, an EEPROM or the like, and stores computer programs that are executed by the processing part 101 and data that are used thereby. The ROM 103 stores a boot program that is executed by the processing part 101 when the device 10 is started up, and programs and settings relating to the operation of the hardware of the device 10.

The main storage part 102 is constituted of a RAM (Random access memory) such as an SRAM or DRAM. The main storage part 102 is used to read out the computer programs stored in the ROM 103 and the auxiliary storage part 104. The main storage part 102 is also utilized as a workspace when the processing part 101 executes these computer programs. The main storage part 102 temporarily stores training data or the like acquired via a network, functions of the artificial intelligence model read out by the auxiliary storage part 104, and so on.

The auxiliary storage part 104 is constituted of a hard disk, a semiconductor memory element such as a flash memory, an optical disk, or the like. In the auxiliary storage part 104, various computer programs to be executed by the processing part 101 such as an operating system and application programs, and various setting data for use in executing the computer programs are stored. Specifically, the auxiliary storage part 104 stores operation software (OS) 1041, a training program TP for prediction, a database (DB) AI1 for artificial intelligence models for prediction, an adverse event database (DB) TR1 for storing adverse event data for drugs and/or occurrence frequency data for adverse events and information about adverse events acquired from the drug database 60, and a database (DB) TR2 for indication data for storing indication data for drugs acquired from the drug database 60 in a non-volatile manner. The training program TP performs processing for training an artificial intelligence model as described later in corporation with the operation software (OS) 1041. In the artificial intelligence model database AI1, untrained artificial intelligence models and trained artificial intelligence models for prediction may be stored.

The communication I/F 105 is constituted of a serial interface such as a USB, IEEE1394 or RS-232C, a parallel interface such as an SCSI, IDE or IEEE1284, and an analog interface constituted of a D/A converter, A/D converter or the like, a network interface controller (NIC) and so on. The communication I/F 105, under the control of the processing part 101, receives data from a measurement part 30 or other external devices, and, when necessary, transmits information stored in or generated by the device 10 to the measurement part 30 or to the outside, or displays it. The communication I/F 105 may communicate with the measurement part 30 or other external devices (not shown, e.g., other computers or cloud systems) via a network.

The input I/F 106 is constituted of a serial interface such as a USB, IEEE1394 or RS-232C, a parallel interface such as an SCSI, IDE or IEEE1284, an analog interface constituted of a D/A converter, A/D converter or the like, and so on. The input I/F 106 accepts character input, clicks, sound input or the like from the input part 111. The accepted inputs are stored in the main storage part 102 or the auxiliary storage part 104.

The input part 111 is constituted of a touch panel, keyboard, mouse, pen tablet, microphone or the like, and performs character input or sound input into the device 10. The input part 111 may be externally connected to the device 10 or may be integrated with the device 10.

The output I/F 107 is constituted, for example, of an interface similar to that for the input I/F 106. The output I/F 107 outputs information generated by the processing part 101 to the output part 112. The output I/F 107 outputs information generated by the processing part 101 and stored in the auxiliary storage part 104 to the output part 112.

The output part 112 is constituted, for example, of a display, a printer or the like, and displays measurement results transmitted from the measurement part 30, various operation windows in the device 10, respective items of training data, an artificial intelligence model, and so on.

The media I/F 108 reads out, for example, application software or the like stored in the storage medium 113. The read out application software or the like is stored in the main storage part 102 or the auxiliary storage part 104. Also, the media I/F 108 writes information generated by the processing part 101 into the storage medium 113. The media I/F 108 writes information generated by the processing part 101 and stored in the auxiliary storage part 104 into the storage medium 113.

The storage medium 113 is constituted of a flexible disk, a CD-ROM, a DVD-ROM or the like. The storage medium 113 is connected to the media I/F 108 by a flexible disk drive, a CD-ROM drive, a DVD-ROM drive or the like. An application program or the like for a computer to execute an operation may be stored in the storage medium 113.

The processing part 101 may acquire application software and various settings necessary for control of the device 10 via a network instead of reading them out of the ROM 103 or the auxiliary storage part 104. It is also possible that the application program is stored in an auxiliary storage part of a server computer on a network and the device 10 accesses this server computer to download the computer program and stores it in the ROM 103 or the auxiliary storage part 104.

Also, in the ROM 103 or the auxiliary storage part 104, an operation system that provides a graphical user interface environment, such as Windows (trademark) manufactured and sold by Microsoft Corporation in the United States, has been installed. The training program TP shall operate on the operating system. In other words, the device 10 may be a personal computer or the like.

### 2-3. Processing by Training Program for Prediction

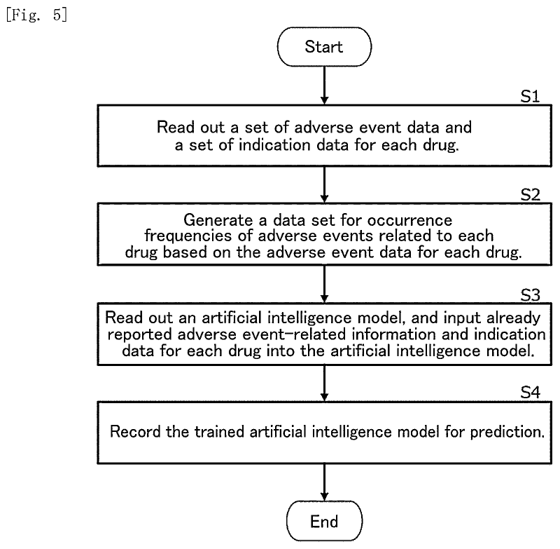

Referring to FIG. 5, the flow of processing for training an artificial intelligence model for prediction is described.

The processing part 101 accepts a command to start processing input by an operator through the input part 111, and, in step S1, reads out a set of adverse event data and a set of indication data for each drug from the database TR1 and the database TR2, respectively, stored in the auxiliary storage part 104.

In step S2, when necessary, the processing part 101 generates a data set for occurrence frequencies from the set of adverse event data for each drug. The method for calculating an occurrence frequency is as described in Section 1.(3) above.

In step S3, the processing part 101 generates already reported adverse event-related information for each drug according to the method described in Section 2-1. above. Also, the processing part 101 reads out an artificial intelligence model from the artificial intelligence model database All stored in the auxiliary storage part 104, and inputs the generated already reported adverse event-related information and a set of indication data linked with the generated adverse events into the artificial intelligence model to train the artificial intelligence model. Here, the artificial intelligence model read out in step S3 may be an artificial intelligence model that has not been trained yet or an artificial intelligence model that has been already trained.

The processing part 101 records the trained artificial intelligence model for prediction into the auxiliary storage part 104 in step S4, and terminates the processing.

The training of an artificial intelligence model for prediction can be carried out using, for example, software such as Python.

### 3. Generation of Test Data for Prediction

Generation of test data for prediction that is input into an artificial intelligence model for prediction is described using adverse events as an example.

### 3-1. Generation of Training Data for Estimation for Training Artificial Intelligence Model for Estimation

### (1) Generation of First Training Data Set

A first training data set may be constituted of a set of data indicating the behavior of a biomarker in one organ or each of multiple different organs. The one organ or multiple different organs may be collected from respective non-human animals to which multiple known drugs with known effects on humans have been individually administered. The first training data set may be stored as a database.

Each item of data indicating the behavior of a biomarker in each organ may be linked with information about the name of a known drug administered, information about the name of an organ collected, information about the name of a biomarker or the like. The term “information about the name” may be a label of the name itself, an abbreviated name or the like, or a label value corresponding to each name.

Each item of data included in the set of data indicating the behavior of a biomarker serves as an element that constitutes a matrix in a first training data set for an artificial intelligence model as described later. When the biomarker is transcriptome, the expression level of each RNA corresponds to data, and serves as an element of a matrix constituting the first training data set. For example, when the biomarker is transcriptome, a loge (fold) value of each known drug obtained by DESeq2 analysis may be used as each element of the first training data set.

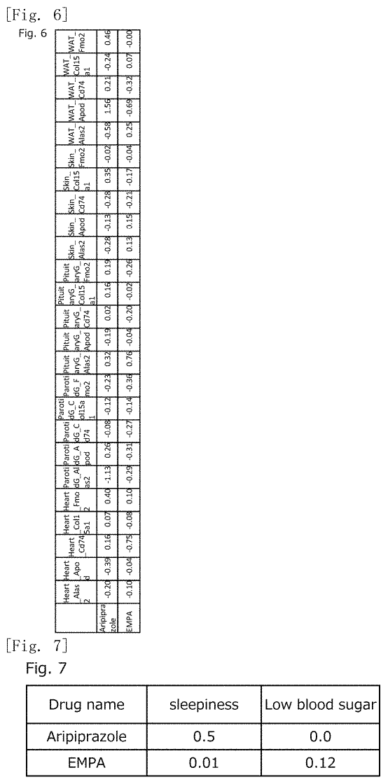

FIG. 6 shows a part of an example of a first training data set in the case where transcriptome is used as a biomarker. The data indicating the behavior of a biomarker is represented as a matrix in which labels each indicating a combination of an organ name and a gene name (which may be represented as “organ-gene”) are aligned in the column direction for each label of the name of a known drug (row direction). Each element of the matrix is the expression level of a gene, which indicated in a column label, in the organ, which is indicated in a column label, collected from non-human animals to which the known drug, which is indicated by a row label, has been administered. More specifically, in the row direction, labels of Aripiprazole and EMPA as known drugs are attached. In the column direction, labels of Heart_Alas2, Heart_Apod, ParotidG_Alas2, ParotidG_Apod and so on are attached. “Heart,” “ParotidG” and so on are labels indicating organs such as heart, parotid gland and so on, and “Alas2,” “Apod” and so on are labels each indicating the name of a gene from which RNA is derived. In other words, the label “Heart_Alas2” means “expression of Alas2 gene in the heart.”

The set of data indicating the behavior of a biomarker may be directly used as a first training data set or may be subjected to standardization, dimensionality reduction or the like before being used as a first training data set. An example of a standardization method can be a method to transform data indicating expression differences such that the mean value is 0 and the variance is 1, for example. The mean value in the standardization can be the mean value in each organ, the mean value in each gene, or the mean value of all data. Also, the dimensionality reduction can be achieved by statistical processing such as a principal component analysis. The parent population in performing statistical processing can be set for each organ, for each gene, or for all data. For example, when the biomarker is transcriptome, only the genes having a p-value not greater than a predetermined value relative to a log2 (fold) value of each known drug obtained by DESeq2 analysis may be used as the elements of the first training data set. The predetermined can be 10⁻³ or 10⁻⁴, for example. Preferred is 10⁻⁴.

The first training data set may be updated in response to the update of the known drugs or the addition of new data indicating the behavior of a biomarker.

### (2) Generation of Second Training Data

The second training data may be constituted of information about adverse events in humans acquired for each of multiple known drugs administered to non-human animals to generate the first training data set. An item of second training data corresponds to information about adverse events (such as “headache”) related to one drug. The information about adverse events used as second training data can be generated from adverse event data acquired from the drug database 60 or the like in the same manner as already reported adverse event-related information used as training data for an artificial intelligence model for prediction as described above.

FIG. 7 shows an example of generated second training data. FIG. 7 shows the occurrence frequency of each adverse event calculated based on adverse event data of aripiprazole and EMPA downloaded from FAERS. The adverse events related to each drug may be, as the presence or absence of adverse events, represented, for example, as “1” when a certain adverse event has been observed and as “0” or “−1” when the adverse event has not been observed.

The second training data may be updated in response to the update of the known drugs, the update of the known database, and so on.

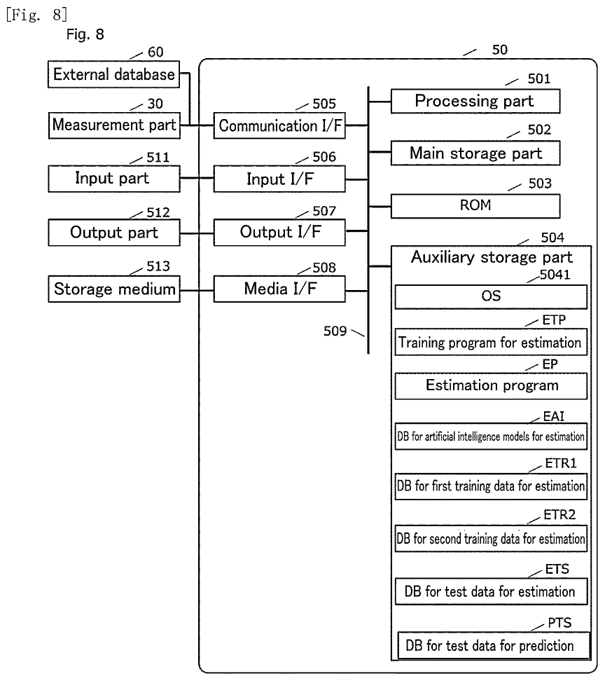

The acquisition of measurement values of a biomarker from a measurement device 30 shown in FIG. 8 is started via a communication I/F 505 by a processing part 501 of a test data generation device 50 when the processing part 501 accepts a request to acquire data from an operator. The acquired measurement values of a biomarker are recorded in a database (DB) ETR1 for first training data for estimation of an auxiliary storage part 504 shown in FIG. 8 by the processing part 501.

The acquisition of adverse event data or a set of adverse event data from the drug database 60 shown in FIG. 8 is started via the communication I/F 505 by the processing part 501 of the test data generation device 50 when the processing part 501 accepts a request to acquire data from the operator. The adverse event data and the set of adverse event data acquired are stored in a database (DB) ETR2 for second training data for estimation stored in the auxiliary storage part 504 by the processing part 501.

### 3-2. Generation of Test Data for Estimation to be Input Into Artificial Intelligence Model for Estimation

The test data for estimation that is input into an artificial intelligence model for estimation to estimate adverse events related to a drug of interest is a data set indicating the behavior of a biomarker in one or more organs of non-human animals to which a drug or the like of interest has been administered as a test substance. The test data for estimation is generated in the same manner as the first training data and stored in a database (DB) ETS for test data for estimation shown in FIG. 8.

### 3-3. Training of artificial intelligence model for estimation and estimation of adverse events

An artificial intelligence model is trained using a first training data set and second training data or a second training data set as described above to construct an artificial intelligence model for estimation. The construction of an artificial intelligence model may include training an untrained artificial intelligence model and retraining an artificial intelligence model which has been once trained. A first training data set and/or second training data updated as described above can be used for retraining.

A first training data set and second training data or a second training data set are input in combination as training data into an artificial intelligence model. In the training data for estimation, the first training data set and the second training data or the second training data set are linked based on (i) labels indicating the names of known drugs administered to non-human animals that are linked with respective data items indicating the behavior of a biomarker in respective organs, which are included in the first training data set, and (ii) labels indicating the names of respective known drugs administered to the non-human animals that are linked with information about adverse events, which are included in the second training data or the second training data set. Based on the label indicating the names of respective known drugs administered to the non-human animals, an artificial intelligence model is trained by associating information about adverse events related to known drugs administered to the non-human animals which is correct (or TRUE, or has a label “1” indicating that it is correct) with the set of data indicating the behavior of a biomarker in respective organs.

Here, when the artificial intelligence model trained to predict each adverse event is an artificial intelligence model of the type in which the algorithm of one artificial intelligence model corresponds to one effect (such as “headache”) such as random forest, SVM, relevance vector machine (RVM), Naive Bayes, AdaBoost, C4.5, stochastic gradient descent (SGD) classifier, Lasso, ridge regression, Elastic Net, SGD regression, or kernel regression, one item of second training data is linked with the first training data set. On the other hand, in the case of an artificial intelligence model that can predict multiple effects (such as “headache,” “vomiting,” . . . ) with one artificial intelligence model such as feed forward neural network, deep leaning or matrix decomposition, the first training data is linked with multiple items of second training data, in other words, a second training data set.

When description is made taking FIG. 6 and FIG. 7 as examples, each row in which a label of each known drug shown in FIG. 6 is shown is respectively linked with each cell shown in FIG. 7 to generate one set of training data to be input into an artificial intelligence model. In other words, the row of Aripiprazole shown in FIG. 6 and “sleepiness-0.5” in the row of Aripiprazole shown in FIG. 7 are linked as one data set. Also, the row of Aripiprazole shown in FIG. 6 and “Low blood sugar-0.0” in the row of Aripiprazole shown in FIG. 7 are linked as one data set. Further, the row of EMPA shown in FIG. 6 and “sleepiness-0.01” in the row of EMPA shown in FIG. 7 are linked as one data set. The row of EMPA shown in FIG. 6 and “Low blood sugar-0.12” in the row of EMPA shown in FIG. 7 are linked as one data set. In other words, from the data of the example in FIG. 6 and FIG. 7, a total of four data sets are generated as training data. Here, 0.5, 0.0, 0.01 and 0.12 in FIG. 7 are occurrence frequencies of the adverse events (with the maximum value being 1).

### 3-4. Device for Generating Test Data for Prediction

An artificial intelligence model for estimation can be constructed using, for example, a device 50 for generating test data for prediction as described below.

In the description of the device 50 for generating test data for prediction and operation of the device 50 for generating test data for prediction, for the same terms as those described in “Overviews of training method and prediction method, and description of terms” and “Generation of training data for estimation for training artificial intelligence model for estimation” above, the above description is incorporated here.

The device 50 for generating test data for prediction (which may be hereinafter referred to as “device 50”) includes at least the processing part 501 and a storage part. The storage part is constituted of a main storage part 502 and/or an auxiliary storage part 504.

FIG. 8 illustrates a hardware configuration of the device 50. The device 50 may be connected to an input part 511, an output part 512, and a storage medium 513. Also, the device 50 may be connected to a measurement part 30, which is a next-generation sequencer, mass spectrometer or the like. In other words, the device 50 may constitute a system for generating test data for prediction connected to a measurement part 30 directly or via a network or the like.

The device 50 basically has the same hardware configuration as the training device 10. Thus, the description in Section 2-2. above is incorporated here. In the device 50, the processing part 501, the main storage part 502, and a ROM (read only memory) 103, the auxiliary storage part 504, the communication interface (I/F) 505, an input interface (I/F) 506, an output interface (I/F) 507, and a media interface (I/F) 508 are connected for mutual data communication by a bus 509.

However, in the auxiliary storage part 504, operation software (OS) 5041, a training program ETP for estimation, a database (DB) EAI for artificial intelligence models for estimation, a database (DB) ETR1 for first training data for estimation, a database (DB) ETR2 for second training data for estimation, a database (DB) ETS for test data for estimation, a database (DB) PTS for test data for prediction are stored in place of the operation software (OS) 1041, the training program TP for prediction, the artificial intelligence model database (DB) AD, the adverse event data database (DB) TR1, and the indication data database (DB) TR2. The database (DB) EAI for artificial intelligence models for estimation stores untrained and trained artificial intelligence models. The database (DB) ETR1 for first training data for estimation stores, as first training data, a set of data indicating the behavior of a biomarker in each organ collected from non-human animals to which each known drug has been administered with labels indicating the names of the drugs administered linked with it. The database (DB) ETR2 for second training data for estimation stores information about adverse events that is used as second training data corresponding to each known drug administered to non-human animals with labels indicating the drug names linked with it. The database (DB) ETS for test data for estimation stores data indicating the behavior of a biomarker in each organ collected from non-human animals to which a drug or the like of interest has been administered as a test substance that are used as test data for estimation.

### 3-5. Processing by Training Program for Estimation

The device 50 provides a training function when the processing part 501 executes the training program ETP for estimation as application software.

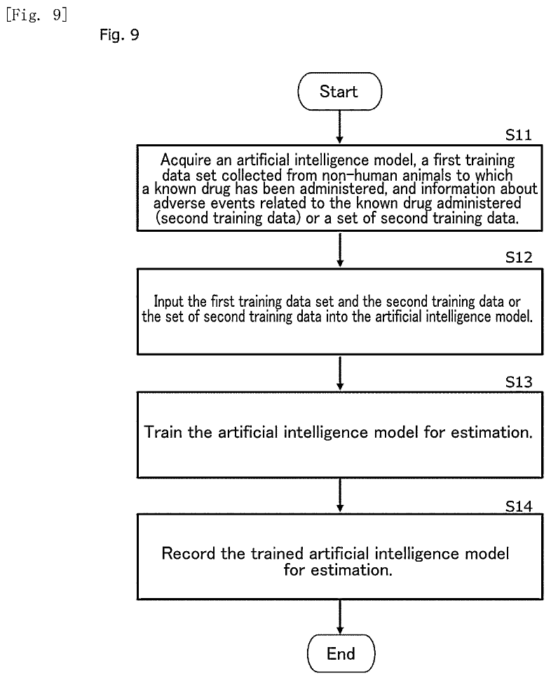

Referring to FIG. 9, the processing that is executed by the training program ETP for estimation is described.

In step S11, the processing part 501 accepts a request to start processing input by an operator through the input part 511, and temporarily reads out an artificial intelligence model stored in the database EAI for artificial intelligence for estimation of the auxiliary storage part 504, for example, into the main storage part 502. Also, the processing part 501 accepts a request to acquire training data input by the operator through the input part 511, and reads out a first training data set acquired from non-human animals to which each known drug has been administered as described in Section 3-1. above from the database ETR1 for first training data for estimation. Further, the processing part 501 reads out information about adverse events corresponding to the administered drugs or a set of such information from the database ETR2 for second training data for estimation as second training data or a set of second training data.

In step S12, the processing part 501 links the first training data set and the second training data or the set of second training data read out in step S11 by means of labels indicating the names of known drugs administered to non-human animals that are linked with the first training data set and labels indicating the names of known drugs administered to non-human animals that are linked with the second training data, and inputs them into an artificial intelligence model.

Next, in step S13, the processing part 501 calculates a parameter such as a weight in a function of the artificial intelligence model to train the artificial intelligence model.

Next, in step S14, the processing part 501 stores the trained artificial intelligence model as an artificial intelligence model for estimation in the database EAI for artificial intelligence for estimation.

The training processing can be performed using, for example, software such as Python.

### 3-6. Processing by Estimation Program

The device 50 generates test data for prediction when the processing part 501 executes the estimation program EP as application software.

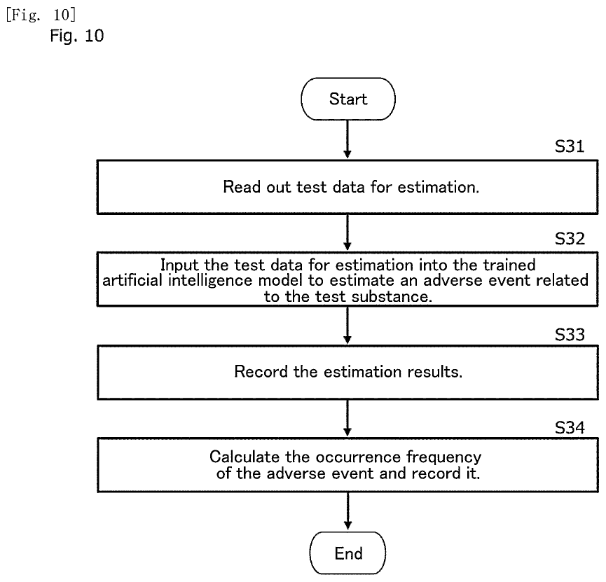

Referring to FIG. 10, the processing that is executed by the estimation program ETP is described.

The processing part 501 accepts a command to start processing input by the operator through the input part 511, and, in step S31 of FIG. 10, reads out test data for estimation from the database ETS for test data for estimation stored in the auxiliary storage part 504. Also, the processing part 501 reads out a trained artificial intelligence model for estimation from the database EAI for artificial intelligence models for estimation stored in the auxiliary storage part 504.

Next, the processing part 501 accepts a command to start prediction input by the operator through the input part 511, and, in step S32, inputs the test data for estimation into the trained artificial intelligence model for estimation to acquire an estimation result about an adverse event related to the drug or the like of interest. The estimation result may be output as a combination of a label indicating an adverse event name and a label indicating whether or not being an adverse event from the trained artificial intelligence model for estimation. As a label indicating whether or not being an adverse event, “1” can be output when the artificial intelligence model estimated that the drug or the like of interest “has” the corresponding adverse event and “0” or “−1” can be output when the artificial intelligence model estimated that the drug or the like of interest “does not have” the corresponding adverse event. For example, when the adverse event is “sleepiness,” “sleepiness:1” is output as an estimation result when it is estimated that the drug or the like of interest has sleepiness. Also, “sleepiness:0” or “sleepiness:−1” is output as an estimation result when it is estimated that the drug or the like of interest does not have sleepiness.

Next, the processing part 501 accepts a command to record the estimation result input by the operator through the input part 511, and, in step S33, records the estimation result estimated in step S32 into the database PTS for test data for prediction in the auxiliary storage part 504.

Next, the processing part 501 accepts a request to start calculation of occurrence frequency input by the operator through the input part 511, and, in step S34, calculates the occurrence frequency of each adverse event corresponding to the drug or the like of interest from which the estimation result has been acquired, and records it as occurrence frequency data for each adverse event related to each drug into the database PTS for test data for prediction in the auxiliary storage part 504. The method for calculating the occurrence frequency is as described in Section 1. above. The occurrence frequency data for each adverse event related to each drug or the like of interest will be test data for prediction.

After step S34, the processing part 501 may accept a command to output input by the operator through the input part 511 or may be triggered by the completion of step S34 to output the estimation result to the output part 512.

The estimation processing can be performed by, for example, using software such as Python.

### 4. Prediction of Indication by Artificial Intelligence Model for Prediction

Prediction of an indication is described using adverse events as an example.

In the description of a device 20 and operation of the device 20, for the same terms as those described in Sections 1. and 2-1. above, the above description is incorporated here.

### 4-1. Acquisition and Recording of Test Data and Trained Artificial Intelligence Model for Prediction

The prediction device 20 may acquire a trained artificial intelligence model for prediction from the artificial intelligence database All recorded in the auxiliary storage part 104 of the device 10 described in FIG. 4 via a network or a storage medium 213 and record it in a database TS1 in the auxiliary storage part 204 of the prediction device 20.

The test data for prediction is acquired from the database PTS for test data for prediction stored in the device 50 for generating test data for prediction described in FIG. 8 via a network or the storage medium 213 by the prediction device 20, and the test data for prediction acquired is recorded into a database TS1 for test data (which may be hereinafter also referred to simply as “database TS1”) stored in the auxiliary storage part 204 by the processing part 201.

### 4-2. Device for Predicting Indication

The prediction of an indication can be achieved using, for example, the prediction device 20 (which may be hereinafter referred to simply as “device 20”).

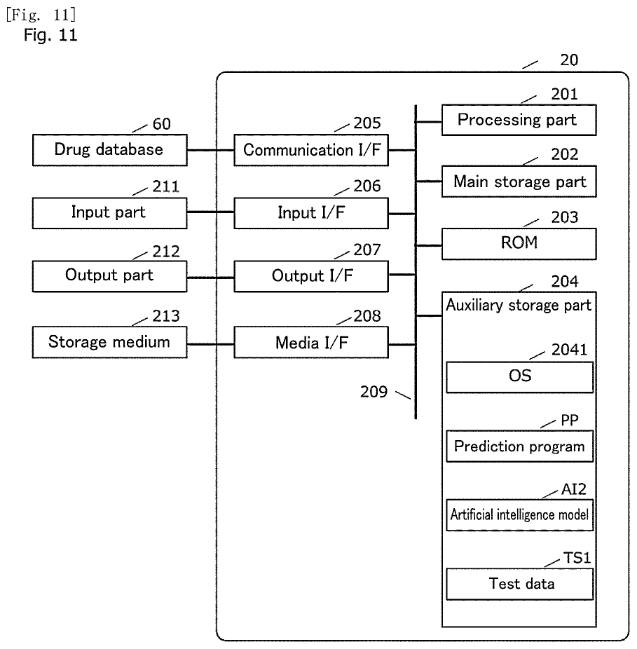

FIG. 11 illustrates a hardware configuration of the prediction device 20 (which may be hereinafter referred to also as “device 20”). The device 20 includes at least the processing part 201 and a storage part. The storage part is constituted of a main storage part 202 and/or an auxiliary storage part 204. The device 20 may be connected to an input part 211, an output part 212, and a storage medium 213. Also, the device 20 is communicably connected to a drug database 60 such as FAERS, all drug labels of DAILYMED, Medical Subject Headings, Drugs@FDA, International Classification of Diseases, or clinicaltrials.gov. Further, the device 20 may be communicably connected to the device 10 and the device 50 via a network.

In the device 20, the processing part 201, the main storage part 202, a ROM (read only memory) 203, the auxiliary storage part 204, a communication interface (I/F) 205, an input interface (I/F) 206, an output interface (I/F) 207, and a media interface (I/F) 208 are connected for mutual data communication by a bus 209.

Because the device 20 has the same basic hardware configuration as the device 10, the description in Section 2-2. above is incorporated here.

However, in the auxiliary storage part 204 of the device 20, operation software (OS) 2041, a prediction program PP, an artificial intelligence model database AI2 for storing a trained artificial intelligence model, and a database TS1 for storing test data for prediction are stored in a non-volatile manner in place of the operation software (OS) 1041, the training program TP for prediction, the artificial intelligence model database AIL the adverse event data database TR1 and the indication data database TR2. The prediction program PP performs processing for predicting an indication as described later in cooperation with the operation software (OS) 2041.

### 4-3. Processing for Predicting Indication

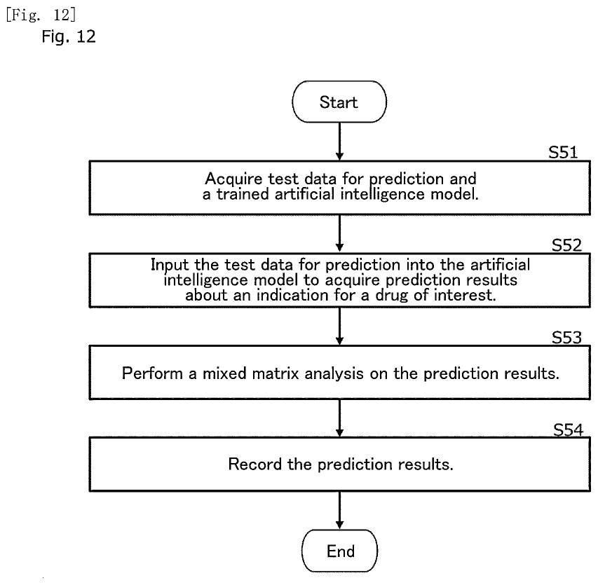

Referring to FIG. 12, the flow of processing for predicting an indication is described.

The processing part 201 accepts a command to start processing input by an operator through an input part 211, and, in step S51 of FIG. 12, read outs test data for prediction from the database TS1 stored in the auxiliary storage part 204. Also, the processing part 201 reads out a trained artificial intelligence model for prediction from the artificial intelligence model database AI2 stored in the auxiliary storage part 204.

Next, the processing part 201 accepts a command to start prediction input by the operator through the input part 211, and, in step S52, inputs the test data for prediction into the trained artificial intelligence model for prediction to acquire prediction results about an indication for a drug or the like of interest. A prediction result may be output from the trained artificial intelligence model as a combination of a label indicating an indication name with a label indicating whether or not the indication is an indication for a drug of interest. As a label indicating whether or not the indication is an indication for the drug or the like of interest, “1” can be output when the drug of interest is predicted to be “effective” against the corresponding indication by the artificial intelligence model and “0” or “−1” can be output when it is predicted to be “ineffective.” For example, when the indication is “Nerve injury” and when the drug or the like of interest is predicted to be effective against nerve injury, “Nerve injury: 1” is output as a prediction result. When the drug or the like of interest is predicted to be ineffective against nerve injury, “Nerve injury: 0” or “Nerve injury: −1” is output as a prediction result. The processing part 201 records these prediction results into the auxiliary storage part 204.

Next, when the test substance is a known drug or an equivalent substance of a known drug, the processing part 201 accepts a command to analyze prediction results input by the operator through the input part 211, and, in step S54, performs a mixed matrix analysis on the prediction results acquired in step S53 to determine whether the prediction result for an indication output for each drug is true positive (TP) or false positive (FP). When the result is true positive, a label “1” is attached to the label indicating the indication name, for example. When the result is false positive, a label “0” is attached to the label indicating the indication name, for example. True positive means that the indication is registered as an “indication” (against which the drug is effective) for each drug registered in the drug database 60, and is also predicted as an “indication” therefor in a prediction result. False positive means that the indication is not registered as an “indication” for each drug registered in the drug database 60 but is predicted as an “indication” in a prediction result. The indication determined to be false positive will be a new indication for the drug or the like of interest. Specifically, the indication data for each drug has a label indicating an indication name and a label indicating whether or not each drug is effective against the indication attached thereto. For example, when the prediction result is “Nerve injury: 1” even though the indication data is “Nerve injury: 0” or “Nerve injury: −1,” the indication can be determined as being false positive. When the indication data is “Nerve injury: 1” and the prediction result is “Nerve injury: 1,” the indication is true positive. Step S54 is not performed on a drug for which no adverse event has been reported.

Next, the processing part 201 accepts a command to record the analysis results input by the operator through the input part 211, and in step S55, records the prediction results acquired in step S53 or analysis results acquired in step S54 into the auxiliary storage part 204 and then terminates the processing.

After step S55, the processing part 201 may accept a command to output input by the operator through the input part 211 or may be triggered by the completion of step S55 to output the analysis results to the output part 212.

The prediction processing can be carried out using, for example, software such as Python. The mixed matrix analysis can be carried out using, for example, software “R.”

### 5. Estimation of Mechanism of Action Mechanism

It is important in developing a new and more effective drug to know the action mechanism by which each drug is effective against a newly predicted indication for each drug.

The test data for prediction used in Section 4. above is acquired based on the behavior of a biomarker in one or more organs in response to the administration of a drug or the like of interest as a test substance to non-human animals. The relationship between the test data for prediction of each test substance and each indication corresponding to each drug or the like of interest can be replaced by the relationship between the behavior of a biomarker in multiple organs in response to the administration of each test substance and each indication. Then, the relationship between the behavior of a biomarker in one or more organs in response to the administration of each test substance and each indication can be linked with a biological reaction by executing a known pathway analysis. The biological reaction can be represented as an information transfer pathway (which is hereinafter referred to simply as “pathway”). Examples of the pathway analysis include KEGG pathway enrichment analysis, REACTOME pathway analysis, and so on.

### 5-1. Device for Estimating Action Mechanism

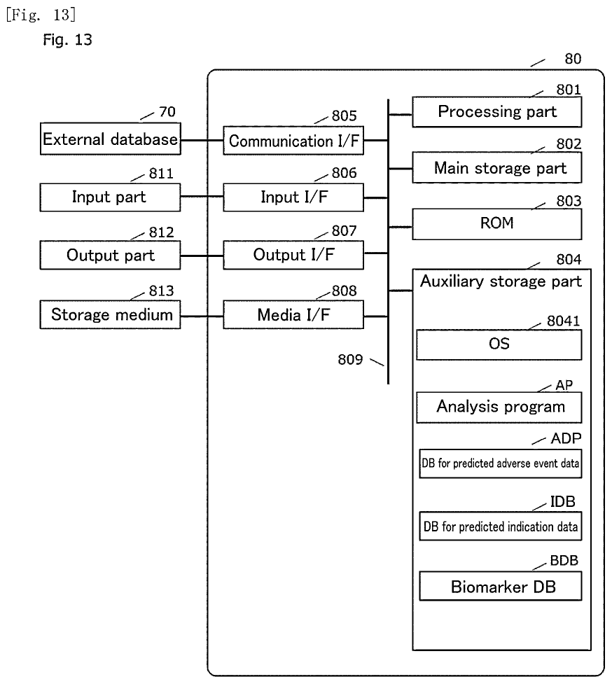

FIG. 13 shows a hardware configuration of a device 80 for estimating an action mechanism (which may be hereinafter referred to also as “device 80”).

Because the device 80 has the same basic hardware configuration as the device 10, the description in Section 2-2. above is incorporated here.

The device 80 includes at least a processing part 801 and a storage part. The storage part is constituted of a main storage part 802 and/or an auxiliary storage part 804. The device 80 may be connected to an input part 811, an output part 812, and a storage medium 813. Also, the device 80 is communicably connected to a pathway database 70 for KEGG pathway enrichment analysis, REACTOME pathway analysis or the like. Further, the device 80 may be communicably connected to the device 10, the device 20 and the device 50 via a network.

In the device 80, the processing part 801, the main storage part 802, a ROM (read only memory) 803, the auxiliary storage part 804, a communication interface (I/F) 805, an input interface (I/F) 806, an output interface (I/F) 807 and a media interface (I/F) 808 are connected for mutual data communication by a bus 809.

However, in the auxiliary storage part 804 of the device 80, operation software (OS) 8041, an analysis program AP for executing a pathway analysis, a database (DB) ADP for predicted adverse event data, a database (DB) IDB for predicted indication data, and a biomarker database (DB) BDB are stored in place of the operation software (OS) 1041, the training program TP for prediction, the artificial intelligence model database All, the adverse event data database TR1 and the indication data database TR2.

The database ADP for predicted adverse event data stores the estimation result about adverse events for each drug obtained in step S32 as described in Section 3-5. above, or the occurrence frequency data for adverse events for each drug calculated in step S34 in association with the name of each drug. The estimation result about adverse events for each drug can be acquired from the database PTS for test data for prediction stored in the device 50 via the communication I/F 805 or the storage medium 813 and recorded in the database ADP for predicted adverse event data of the auxiliary storage part 804 by the device 80.

The database IDB for predicted indication data stores the prediction result about indications for each drug obtained in step S52 as described in Section 4-3. above in association with the name of each drug. The prediction result about indications for each drug can be acquired from the auxiliary storage part 204 of the device 20 via the communication I/F 805 or the storage medium 813 and recorded in the database IDB for predicted indication data of the auxiliary storage part 804 by the device 80.

The biomarker database BDB stores the test data for estimation as described in Section 3-2. above in association with the name of each drug. The test data for estimation can be acquired from the database ETS for test data for estimation stored in the device 50 via the communication I/F 805 or the storage medium 813 and recorded in the biomarker database BDB in the auxiliary storage part 804 by the device 80.

The analysis program AP may include a software R package “clusterProfiler” or the like when KEGG pathway enrichment analysis, for example, is performed. Also, when REACTOME pathway analysis is performed, the analysis program AP may include browser software for accessing https://reactome.org/ or the like.

### 5-2. Processing by Analysis Program

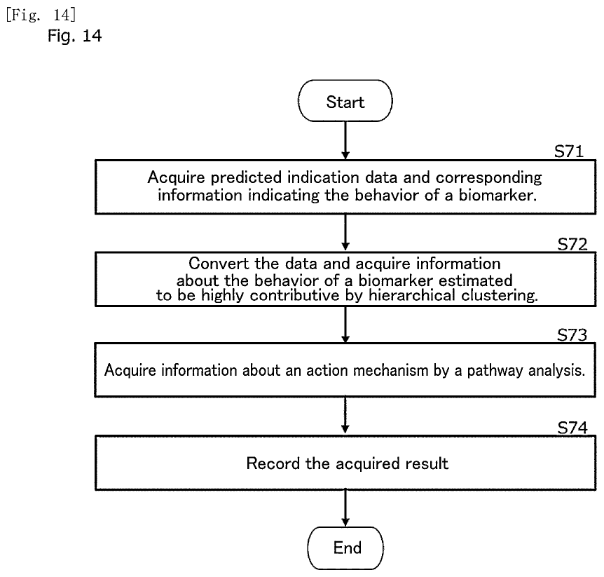

Referring to FIG. 14, the flow of analytical processing for estimating the mechanism by which each drug acts on a new indication is described.

The processing part 801 accepts a command to start data acquisition input by an operator through the input part 811, and, in step S71 shown in FIG. 14, reads out the data on occurrence frequency of adverse events for each drug calculated in step S34 as described in Section 3-5. above from the database ADP for predicted adverse event data. Also, the processing part 801 reads out test data for estimation corresponding to each drug from the biomarker database BDB.

In step S72, the processing part 801 accepts a command to start processing input by the operator through the input part 811, and convers the estimation result about adverse events for each drug and the test data for estimation read out in step S71 into binary matrix representation. Optionally, the processing part 801 may perform a principal component analysis or the like on the data converted into binary matrix representation for dimensional transformation of it. The processing part 801 performs hierarchical clustering on the converted data or converted and dimensionally reduced data. This processing can be achieved using, for example, software “R.” By this processing, the behavior of a biomarker that contributed to the prediction of adverse events for each drug can be estimated. These analyses can be carried out using software “R” or the like.

In step S73, the processing part 801 accepts a command to start a pathway analysis input by the operator through the input part 811, and, inputs the behavior of a biomarker estimated to be highly contributive by hierarchical clustering in step S72 into a pathway database for KEGG pathway enrichment analysis, REACTOME pathway analysis or the like, and acquires information about which biological information transfer pathway is involved from the pathway database as information about the action mechanism of each drug.

Next, the processing part 801 accepts a command to record the prediction result input by the operator through the input part 811, and, in step S74, terminates the processing after recording the result acquired in step S73 in the auxiliary storage part 804.

The processing part 801 may accept a command to output input by the operator through the input part 811 after step S74, or may be triggered by the completion of step S74 to output the acquired result to the output part 812.

### 6. Computer Programs

### 6-1. Training Program for Prediction

A training program for prediction is a computer program that causes a computer to execute the processing including steps S1 to S4 as described in connection with training of an artificial intelligence model in Section 2. to cause the computer to function as the training device 10.

### 6-2. Prediction Program

A prediction program is a computer program that causes a computer to execute the processing including steps S51 to S54 as described in Section 4. to cause the computer to function as the prediction device 20.

### 6-3. Program for Generating Test Data for Prediction

A program for generating test data for prediction is a computer program that causes a computer to execute the processing including steps S11 to S14 and steps S31 to S34 as described in Section 3. above to cause the computer to function as the test data generation device 50.

### 6-4. Mechanism Estimation Program

A program for mechanism estimation program is a computer program that causes a computer to execute the processing including steps S71 to S74 as described in Section 5. above to cause the computer to function as the action mechanism estimation device 80.

### 7. Storage Medium Having Computer Programs Stored Therein

This disclosure relates to a storage medium having the computer programs as described in Section 6. above stored therein. The computer programs are stored in a storage medium such as a hard disk, a semiconductor memory element such as or flash memory, or an optical disk. Also, the computer programs may be stored in a storage medium connectable via a network such as a cloud server. The computer programs may be program products that are in a downloadable form or stored in a storage medium.

The storage format of the programs in the storage medium is not limited as long as a device as described above can read the programs. The storage in the storage medium is preferably in a non-volatile manner.

### 8. Modifications

In this specification, the same reference numeral attached to hardware indicates the same part or same function.

In Sections 2. and 4. above, an embodiment is shown in which the training device 10 and the prediction device 20 are different computers. However, one computer may perform training of an artificial intelligence model and prediction. Also, the artificial intelligence model database All may be stored on a cloud and accessed when the training and prediction are performed.

In Section 3 above, the test data generation device 50 trains an artificial intelligence model for estimation, and generates test data for prediction using the artificial intelligence model for estimation. However, the training of an artificial intelligence model for estimation and the generation of test data for prediction may be performed by different computers. Also, the generation of test data for prediction, the generation of training data for prediction and the prediction of an indication may be performed by one computer. Also, the artificial intelligence model database All and the database EAI for artificial intelligence models for estimation may be stored on a cloud and accessed when the training and prediction are performed.

In Sections 1. to 4. above, information about adverse events is used for the explanation of training of an artificial intelligence model and indication prediction. However, side effects may be used instead of adverse events. In this case, the term “adverse events” in each device, each processing and each method can be replaced by the term “side effects” except for the definition of the terms.

### 9. Verification of Effects of Artificial Intelligence Model

### 9-1. Evaluation of Performance of Artificial Intelligence Model for Prediction

### (1) Training of Artificial Intelligence Model, and Evaluation of Performance of Trained Artificial Intelligence Model (Reference Example)

For all drugs reported to the U.S. Food & Drug Adverse Event Reporting System (FAERS) from the third quarter of 2014 to the fourth quarter of 2017, all occurrence frequency data for adverse events and all indication data registered for each drug were acquired. There are 11,310 indications. Specifically, for 4,885 drugs, a data set including a set of occurrence frequency data and a set of indication data was acquired.

Using all the data, an SVM was trained for each indication according to the generation of training data as described in Section 2-1. above to generate a trained artificial intelligence model.

Occurrence frequency data for 17,155 adverse events registered for respective 4,885 drugs registered in FAERS was individually calculated to generate a set of occurrence frequency data for adverse events for each drug. The sets of occurrence frequency data for adverse events for respective drugs were individually input as test data into the trained artificial intelligence model to perform prediction of indications.

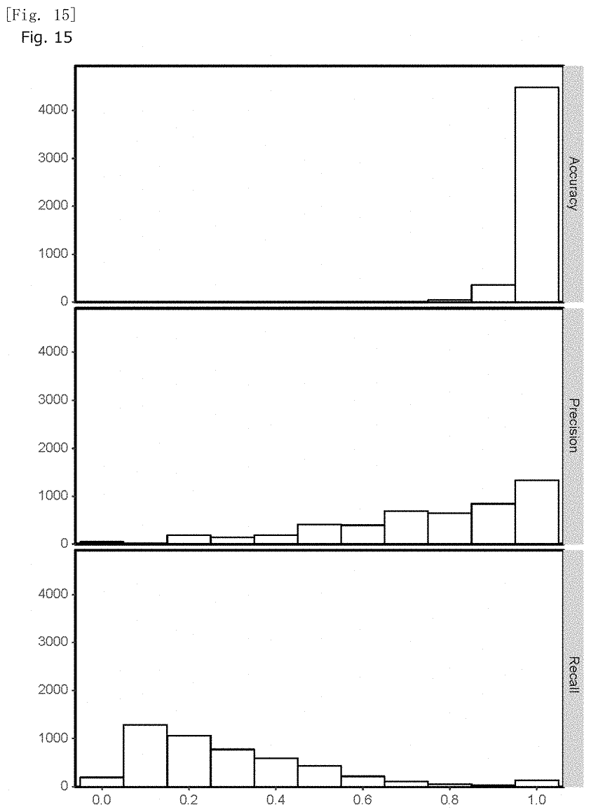

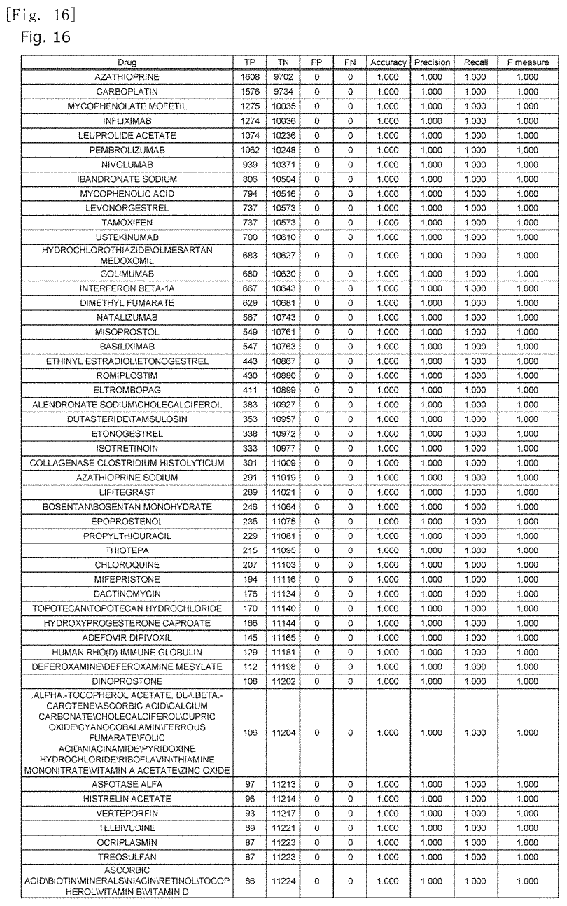

The results are shown in FIG. 15 to FIG. 18. FIG. 15 and FIG. 16 show results showing how accurately the indications reported for respective drugs were able to be predicted.

FIG. 15 shows, for all drugs, the distributions of accuracy score, which indicates the accuracy of prediction, recall score, which indicates the coverage in the case of being predicted as an “indication,” and precision score, which indicates the reliability in the case of being predicted as an “indication” in rod graphs. The accuracy score and the precision score are more accurate as they are closer to 1.0. The correctness of an indication against which the drug is reported to be “effective” is intended to approach 100% as the recall score is closer to 1.

The vertical axes of the graphs show the number of drugs that belong to each quantile when the score ranging from −0.1 to 1.0 is divided into 11 quantiles of 0.1.

For all drugs input as test data into the trained artificial intelligence model, the accuracy score of the results of prediction of indications was as high as not lower than 90% for 4,764 drugs out of 4,885 drugs (97.5%).

Out of 4,885 drugs, 1,790 drugs (36.6% of all drugs) showed a precision score of 90% or higher, 3,252 drugs (66.6% of all drugs) showed a precision score of 70% or higher, and 4,238 drugs (86.8% of all drugs) showed a precision score of 50% or higher.

Out of 4,885 drugs, 746 drugs (15.3% of all drugs) showed a recall score of 50% or higher, 1,951 drugs (39.9% of all drugs) showed a recall score of 30% or higher, and 4,092 drugs (83.8% of all drugs) showed a recall score of 10% or higher.

FIG. 16 shows respective scores of the top 50 drugs having accuracy, precision and recall scores that are all 1.0 among the 4,885 drugs. In FIG. 8, TN represents true negative, TP represents true positive, FN represents false negative, and FP represents true positive. True negative indicates the number of items that were able to be predicted as not being indications for those that are not indications, and true positive indicates the number of items that were able to be predicted as being indications for those that are indications. False negative indicates the number of items that were predicted as being not indications for those that are indications, and false positive indicates the number of items that were predicted as being indications for those that are not indications. The F-measure score is a harmonic mean between the precision score and the recall score, and is an index for evaluating how much accuracy is obtained when the precision score and the recall score are integrated.

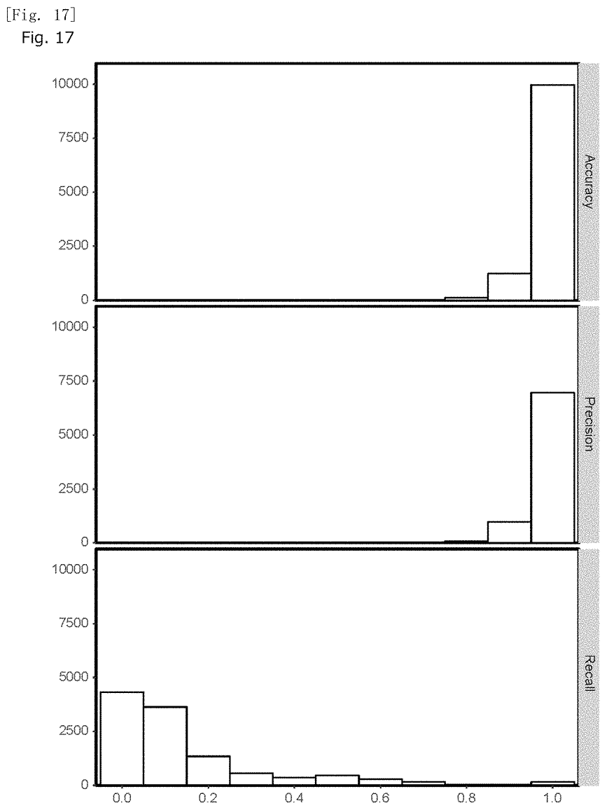

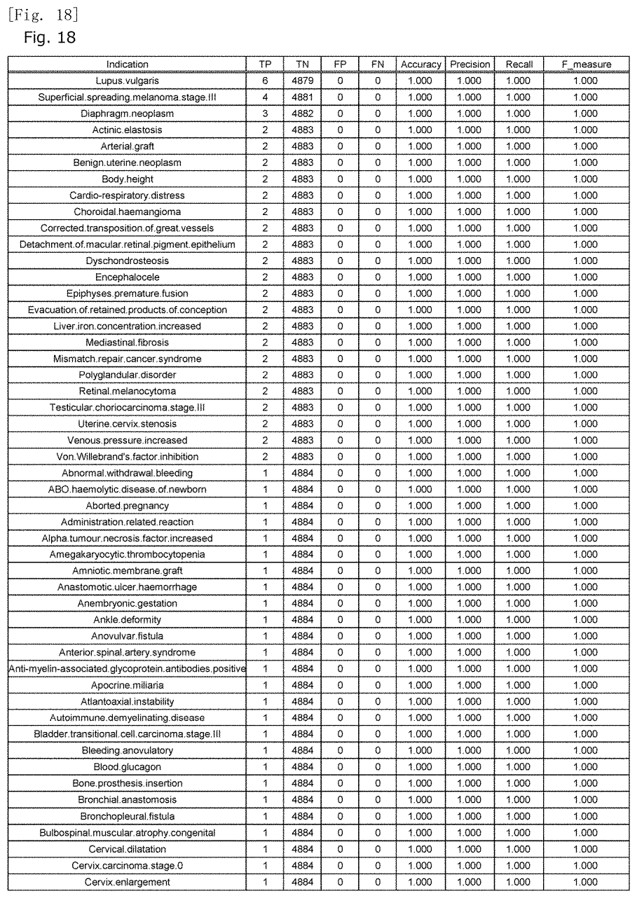

FIG. 17 and FIG. 18 show results showing how accurately the results of prediction of indications derived from the trained artificial intelligence model predicted each indication reported (registered in FAERS).

FIG. 17 shows, for all indications, the distributions of accuracy score, recall score, and precision score in rod graphs. The configuration of the graphs is the same as FIG. 15.

For all reported indications, the accuracy score of the prediction results was as high as not lower than 90% for 10,929 indications out of 11,310 indications (96.6%).

Out of 11,310 indications, 7,230 indications (63.9% of all TIs) showed a precision score of 90% or higher, and 8,016 indications (70.9% of all TIs) showed a precision score of 80% or higher.

Out of 11,310 indications, 972 indications (8.6% of all TIs) showed a recall score of 50% or higher, 1,786 indications (15.8% of all TIs) showed a recall score of 30% or higher, and 4,873 indications (43.1% of all TIs) showed a recall score of 10% or higher.

FIG. 18 shows respective scores of top 50 indications having accuracy, precision and recall scores that are all 1.0 among the 11,310 indications. The terms used in FIG. 18 are the same as those in FIG. 16.

Also, the TN, TP, FN, FP, accuracy score, precision score, recall score, and F-measure score of all indications are shown as FIG. 16 at the end of Detailed Description of the Invention.

The above evaluation results indicate that the trained artificial intelligence model disclosed in this specification can predict indications from information about adverse events.

### (2) Blind Evaluation Using Trained Artificial Intelligence Model

Next, it was evaluated whether accurate prediction can be made using information about adverse events that are not included in a set of training data.

The drugs used for training of an artificial intelligence model in Section 7.(1) above include drugs approved by U.S. Food and Drug Administration (FDA) and/or Pharmaceuticals and Medical Devices Agency (PMDA) from 2017 to 2019, and 61 drugs reported by repositioning by Perwitasari et al., (2013): Pharmaceuticals (Basel) 6, 124-160.

Thus, in the blind evaluation of an artificial intelligence model, an SVM was trained in the same manner as described in Section 7.(1) above using a set of training data which does not include information about adverse events and a set of indication data of the 61 drugs.

Next, the information about adverse events related to the 61 drugs was input into the trained artificial intelligence model, and prediction of indications was performed in the same manner as described in Section 7.(1) above.

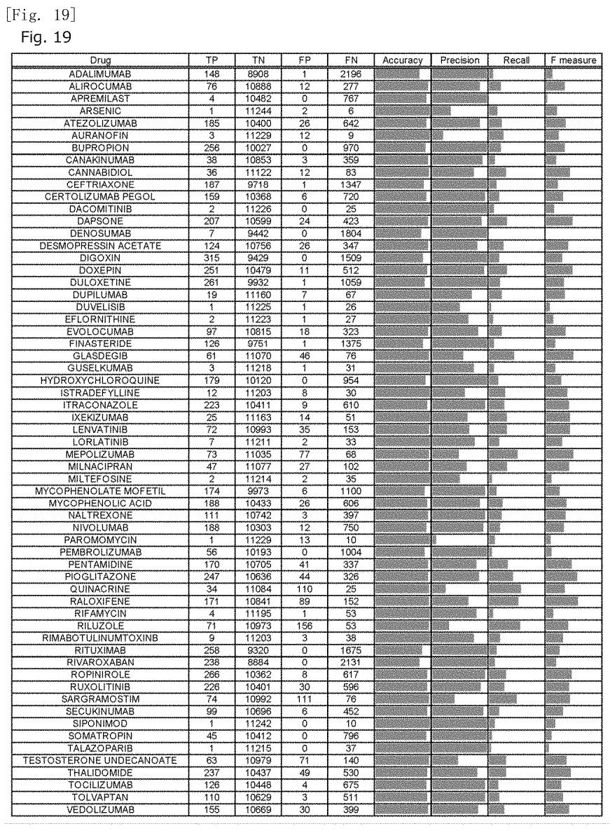

The results are summarized in FIG. 19. The terms used in FIG. 19 have the same meaning as those in FIG. 16.

Out of the 61 drugs, 54 drugs (88.5% of the drugs) showed an accuracy score of 90% or higher. Out of the 61 drugs, 27 drugs (44.3%) showed a precision score of 90% or higher, 44 drugs (72.1%) showed a precision score of 70% or higher, 53 drugs (86.9%) showed a precision score of 50% or higher. Out of the 61 drugs, 4 drugs (6.6%) showed a recall score of 50% or higher, 17 drugs (27.9%) showed a recall score of 30% or higher, and 45 drugs (73.8%) showed a recall score of 10% or higher.

These results indicate that prediction of indications can be made for drugs that are not included in a set of training data with accuracy guaranteed.

### 9-2. Prediction of Indication Using Estimated Test Data for Prediction

### (1) Evaluation by Cross-Validation

Using an RF as an artificial intelligence model instead of an SVM used in Section 9-1. above, an artificial intelligence model for prediction was trained in the same manner as in Section 9-1. For training of the RF, ‘RandomForestClassifier( )’ (Python package ‘scikit-learn’) was used. In ‘RandomForestClassifier( )’, parameter ‘n_estimator’ was set to minimize the generalization error. The other parameters were set to default.

According to the method described in Section 3. above (the method described in Patent Document 2 and Non-Patent Document 2), test data for predicting adverse events related to 15 types of test drugs (alendronate, acetaminophen, aripiprazole, asenapine, cisplatin, clozapine, doxycycline, empagliflozin, lenalidomide, lurasidone, olanzapine, evolocumab, risedronate, sofosbuvir and teriparatide) was generated. Here, the test data for prediction is referred to as “virtual” AE (V-AE).

For the 15 types of test drugs, the occurrence frequency was calculated for all adverse events registered in FAERS, and linked with a label indicating the name of each drug. Also, for all 15 types of test drugs, indication data was acquired for all indications registered in FAERS and linked with a label indicating the name of each drug. In FAERS, 17,155 adverse events and 11,310 indications have been reported. Here, the information about adverse events related to each drug actually acquired from the drug database is referred to as “real” AE (R-AE).

Also, the first training data for an artificial intelligence model for estimation was acquired for each drug by administering the 15 types of test drugs to mice according to the method described in Non-Patent Document 2. As the second training data, a set of data about occurrence frequency of all adverse events for each drug registered in FAERS was used.

The first training data and the second training data were input into the artificial intelligence model RF to train the artificial intelligence model, whereby an artificial intelligence model for estimation was generated.

Data indicating the behavior of a biomarker of the first training data was input into the trained artificial intelligence model for estimation as test data for estimation to acquire V-AE for each drug as a prediction result.

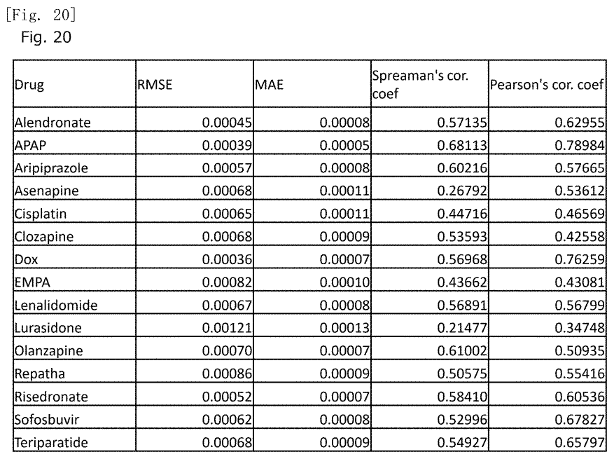

Next, the V-AE and R-AE were compared. The two groups were compared by obtaining a Pearson correlation coefficient and a Spearman's correlation coefficient. The results are shown in FIG. 20. Good correlation was observed for many drugs.

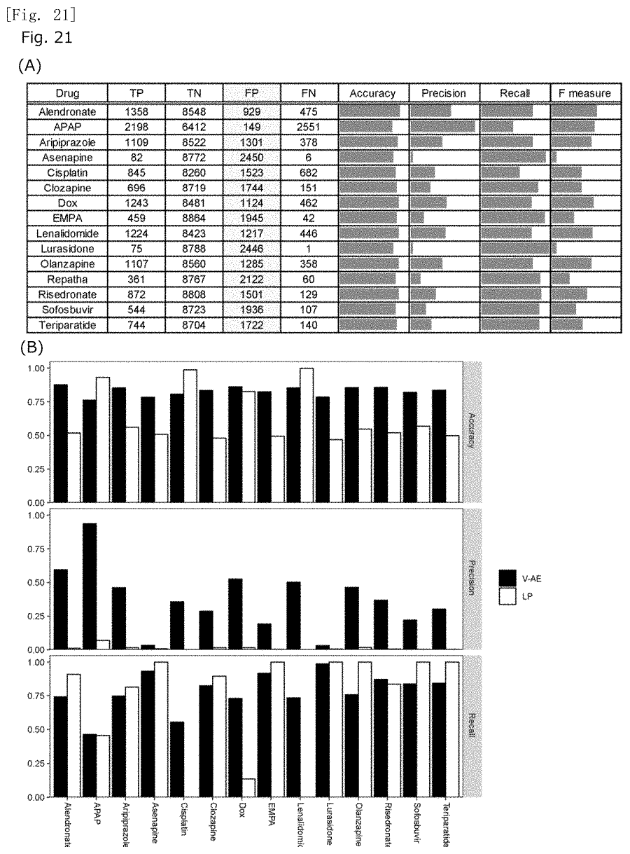

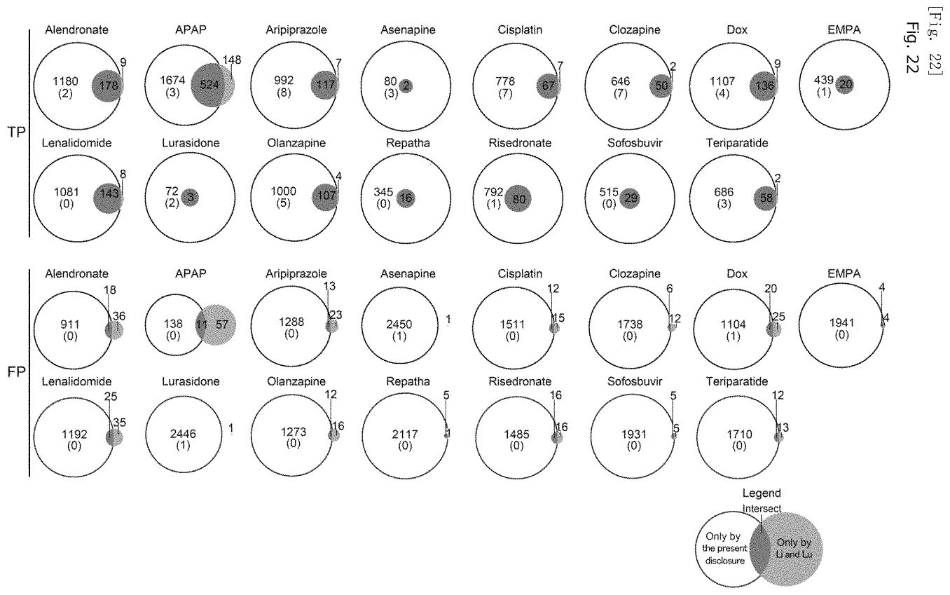

Next, an artificial intelligence model for prediction was trained with the occurrence frequencies of all adverse events related to all drugs registered in FAERS linked with indication data for all the drugs. As the artificial intelligence model, an RF was used. The V-AE was input into the trained artificial intelligence model for prediction to predict indications for the 15 test drugs. The results are shown in FIG. 21(A) as a mixed matrix. The mixed matrix analysis was performed using software “R.” The 15 types of drugs all exhibited a good accuracy score.

In Non-Patent Document 2, a method for predicting an indication for a drug using R-AE as test data and link prediction (LP) as an artificial intelligence model is described. Thus, comparison was made between the accuracy of prediction by the prediction method using V-AE according to this embodiment and the accuracy of prediction by the method using LP as described in Non-Patent Document 2. The results are shown in FIG. 21(B).

The accuracy score and the recall score were good for both the prediction method using V-AE and the method using LP. On the other hand, the prediction score was significantly improved for the prediction method using V-AE for all the 15 types of test drugs. This indicates that the prediction method using V-AE is more accurate.

### (2) Comparison with Prior Art

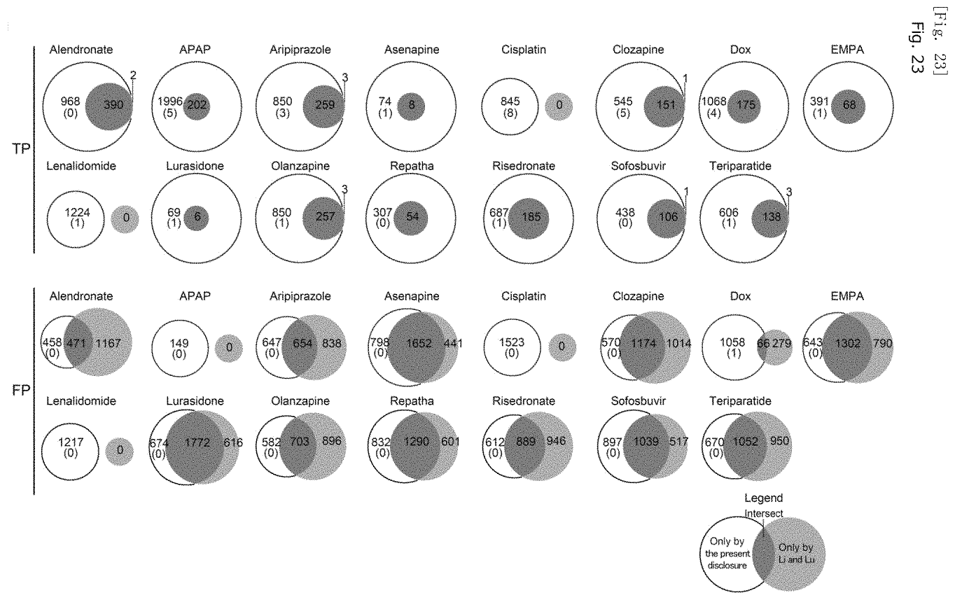

Comparison was made between the results of prediction of indications by the prediction method using V-AE and the prediction method using R-AE (the One-Class SVM method described in Non-Patent Document 2). First, comparison was made between the results of prediction of indications by V-AE and the results of prediction of indications by R-AE. The results are shown in FIG. 22. The upper part of FIG. 22 shows the results of comparison between the numbers of true positive (TP) indications predicted by the two prediction methods. The lower part shows the results of comparison between the numbers of false positive (FP) indications, namely new indications.

The results of prediction of TP indications using V-AE encompassed the results by the prediction method using R-AE for all test drugs. However, for 2 types of test drugs, the prediction method using R-AE was not able to predict TP indications. This indicates that the prediction method using V-AE is higher in prediction accuracy.

In the comparison of FP indications, the prediction method using V-AE was able to detect much more FP indications than the prediction method using R-AE. This indicates that the prediction method using V-AE can explore candidate indications different from those that can be explored by the prediction method using R-AE.

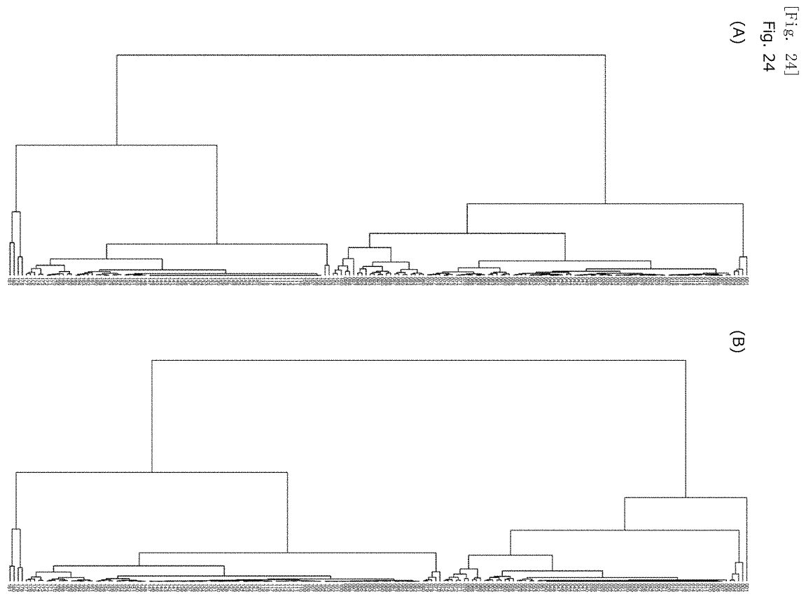

Next, comparison was made of the result of prediction of indications between the prediction method using V-AE and the prediction method using R-AE based on LP as described in Non-Patent Document 2. First, comparison was made between the results of prediction of indications based on V-AE and the results of prediction of indications based on R-AE. The results are shown in FIG. 23. The upper part of FIG. 23 shows the results of comparison between the numbers of true positive (TP) indications predicted by the two prediction methods. The lower part shows the results of comparison between the numbers of false positive (FP) indications, in other words, the numbers of new indications.

The results of prediction of TP indications using V-AE encompassed the results by the prediction method using R-AE for 13 types of test drugs. However, for 2 types of test drugs, the prediction method using R-AE was not able to predict TP indications. This indicates that the prediction method using V-AE is higher in prediction accuracy.

In the comparison of FP indications, the prediction method using V-AE was able to detect FP indications different from those that were able to be detected by the prediction method using R-AE. This indicates that the prediction method using V-AE can explore candidate indications different from those that can be explored by the prediction method using R-AE.

### 9-3. Estimation of Action Mechanism on Indications

By examining a biomarker associated with the estimated indications, it is possible to estimate a mechanism by which a drug acts on the estimated indications.

The occurrence frequency of each V-AE was predicted based on the behavior of a biomarker in one or more organs of mice in response to the administration of each test drug. Thus, for V-AE corresponding to each drug that is important to estimate an indication for each drug, the behavior of a biomarker that contributes to estimation of each V-AE was estimated.

For 14 types of test drugs except repatha (repatha was excluded from the 15 types of test drugs because it is not included in SIDER4.1), characteristics of V-AE that are important for the estimation of 3,054 types of indications reported in both FAERS and SIDER were extracted.

The extraction of characteristics was made by principal component analysis (PCA). The PCA was performed on V-AE and the pattern of transcriptome corresponding to each indication. First, for each indication, binary matrix representation was used to convert the pattern of each V-AE into a transcriptome pattern (1: important AE/organ gene, 0: others). This processing was achieved using software “R.” The PCA was performed on the binary matrix to obtain two principal component scores, PC1 and PC2, for each indication. The PCA was performed using default parameters and using a software “R” function “prcomp.” Hierarchical clustering was performed on the results of the PCA. The hierarchical clustering was performed using the default of a software “R” function “hclust” (Yu et al., 2012, Omics: a journal of integrative biology 16, 284-287).

The relationship between the V-AE and each indication of each test drug on which hierarchical clustering was performed is shown in a tree diagram (FIG. 24(A)). The V-AE is predicted based on a transcriptome profile in multiple organs that depends on the administration of each test drug. Thus, the relationship between the V-AE and each indication of each test drug can be converted into a tree diagram for the relationship between a transcriptome profile in multiple organs in response to the administration of each test drug and each indication (FIG. 24(B)). Then, the relationship between a transcriptome profile in multiple organs in response to the administration of each test drug and each indication can be linked with a biological reaction by performing a known pathway analysis.

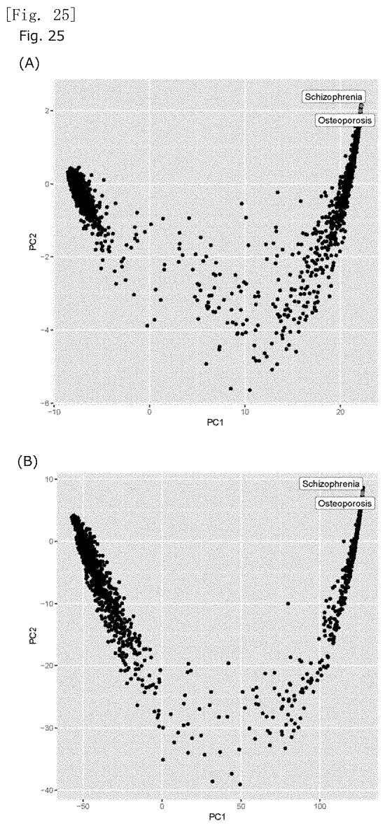

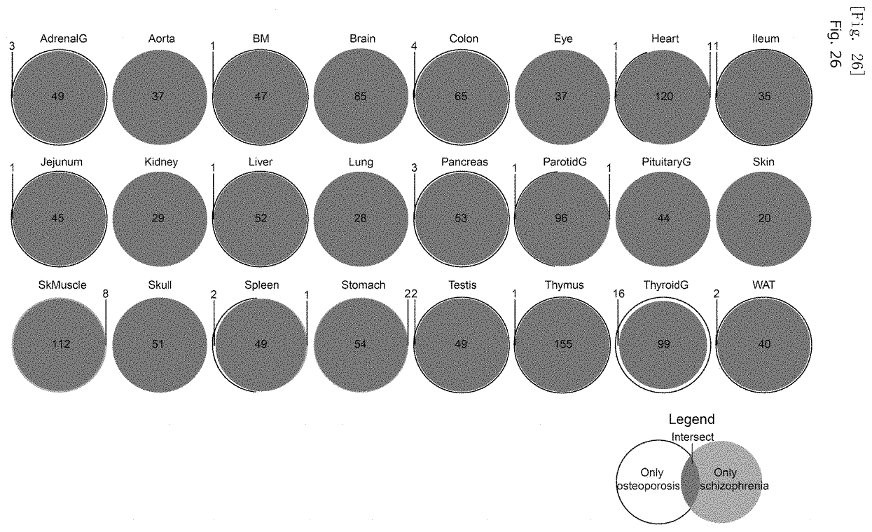

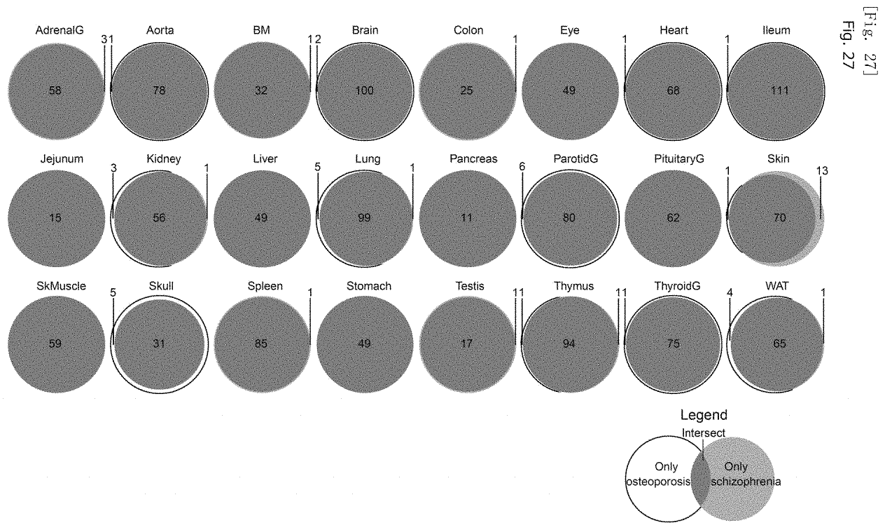

For osteoporosis and schizophrenia, pathway analyses were performed on some of transcriptome profiles in multiple organs in response to the administration of each test drug. As the pathway analyses, KEGG pathway enrichment analysis and REACTOME pathway analysis were performed. REACTOME pathway analysis was performed according to https://reactome.org/. In REACTOME Pathways analysis, it was determined that there was a significant difference when the FDR value was smaller than 0.05. KEGG pathway enrichment analysis was performed using R package “clusterProfiler” version 3.10.1. In KEGG pathway enrichment analysis, it was determined that there was a significant difference when the p-value was smaller than 0.05. It is possible to predict the therapeutic mechanism for each disease from the drugs predicted to be applicable to the treatment of osteoporosis and schizophrenia based on a tree diagram of the PCA result. FIG. 25 shows the distribution of the principal component 1 (PC1) and the principal component 2 (PC2) of the V-AE and transcriptome pattern for osteoporosis and schizophrenia. FIG. 25(A) shows the distribution of the V-AE, and FIG. 25(B) shows the distribution of the transcriptome pattern. The result of a transcriptome analysis after the PCA analysis showed that the action mechanisms of the drugs on osteoporosis and schizophrenia are very similar. For the pathways estimated to be associated with osteoporosis and schizophrenia by the mechanism analysis in this section, comparison was made between the prediction made using REACTOME Pathways and the prediction made using KEGG pathway. FIG. 26 shows the results in the case where REACTOME Pathways was used, and FIG. 27 shows the results in the case where KEGG pathway was used. FIG. 26 and FIG. 27 show the number of pathways estimated for osteoporosis and schizophrenia in each organ in Venn diagrams. The overlapped parts indicate pathways estimated in common for osteoporosis and schizophrenia. FIG. 26 and FIG. 27 also indicate that the pathways for treating osteoporosis and the pathways for treating schizophrenia are very similar.

### DESCRIPTION OF REFERENCE NUMERALS

10: training device
20: prediction device
101: processing part
201: processing part

1. A method for predicting an indication for a drug of interest or its equivalent substance, comprising:
inputting estimated adverse event-related information estimated from a set of data indicating the behavior of a biomarker in one or more organs collected from non-human animals to which the drug of interest or its equivalent substance has been administered as a test substance into an artificial intelligence model for prediction as test data to predict an indication for the drug of interest or its equivalent substance.

2. The prediction method according to claim 1,
wherein the artificial intelligence model for prediction is trained by means of a set of training data, and
wherein the set of training data is data in which (I) already reported adverse event-related information and/or already reported side effect-related information reported for individual known drugs is/are linked with (II) indication data reported for the known drugs.

3. The prediction method according to claim 1,
wherein the artificial intelligence model for prediction corresponds to one indication.

4. The prediction method according to claim 1,
wherein the artificial intelligence model for prediction corresponds to multiple indications.

5. The prediction method according to claim 1,
wherein the estimated adverse event-related information and/or estimated side effect-related information is/are generated using an artificial intelligence model for estimation that is different from the artificial intelligence model for prediction.

6. The prediction method according to claim 1,
wherein the set of training data is generated by linking labels indicating indications for the known drugs and information about adverse events reported for the known drugs with labels indicating the names of the known drugs.

7. The prediction method according to claim 1,
wherein the estimated adverse event-related information and/or estimated side effect-related information correspond(s) to (1) the presence or absence of multiple adverse events and/or side effects, or (2) the occurrence frequencies of multiple adverse events and/or side effects.

8. A device for predicting an indication for a drug of interest or its equivalent substance, comprising a processing part,
wherein the processing part is configured to input estimated adverse event-related information estimated from a set of data indicating the behavior of a biomarker in one or more organs collected from non-human animals to which the drug of interest or its equivalent substance has been administered as a test substance into an artificial intelligence model for prediction as test data to predict an indication for the drug of interest or its equivalent substance.

9. A computer program for predicting an indication for a drug of interest or its equivalent substance, executable by a computer to cause the computer to execute the step of inputting estimated adverse event-related information estimated from a set of data indicating the behavior of a biomarker in one or more organs collected from non-human animals to which the drug of interest or its equivalent substance has been administered as a test substance into an artificial intelligence model for prediction as test data to predict an indication for the drug of interest or its equivalent substance.

10. An estimation method for estimating an action mechanism of a test substance in a living organism, comprising:
hierarchizing the set of data indicating the behavior of a biomarker in one or more organs used in predicting an indication by clustering based on a prediction result about an indication predicted by a prediction method according to claim 1, and
performing a pathway analysis on the hierarchized set of data indicating the behavior of a biomarker to acquire information about an action mechanism of the test substance.

11. An estimation device for estimating an action mechanism of a test substance in a living organism, comprising a processing part,
wherein the processing part is configured to hierarchize the set of data indicating the behavior of a biomarker in one or more organs used in predicting an indication by clustering based on a prediction result about an indication predicted by a prediction method according to claim 1, and to perform a pathway analysis on the hierarchized set of data indicating the behavior of a biomarker to acquire information about an action mechanism of the test substance.

12. An estimation program for estimating an action mechanism of a test substance in a living organism, executable by a computer to cause the computer to execute processing including the steps of:
hierarchizing the set of data indicating the behavior of a biomarker in one or more organs used in predicting an indication by clustering based on a prediction result about an indication predicted by a prediction method according to claim 1, and
performing a pathway analysis on the hierarchized set of data indicating the behavior of a biomarker to acquire information about an action mechanism of the test substance.
